(19) United States
(12) Patent Application Publication
(10) Pub. No.: US 2026/0159903 A1
(43) Pub. Date: Jun. 11, 2026

(54) METHOD FOR PREDICTING THERAPEUTIC RESPONSE OF PHARMABIOTICS AND TREATMENT METHOD OF VARIOUS DISEASES USING THE SAME

(71) Applicant: ENTEROBIOME INC., Goyang-si, Gyeonggi-do (KR)

(72) Inventors: Jae-Gu SEO, Gimpo-si (KR); Moon-Gi HONG, Goyang-si (KR); Dokyung LEE, Seoul (KR); Dohak KIM, Gwangmyeong-si (KR); Da Yeon YOO, Goyang-si (KR); Sungyoon KIM, Goyang-si (KR)

(73) Assignee: ENTEROBIOME INC., Goyang-si, Gyeonggi-do (KR)

(21) Appl. No.: 18/708,914

(22) PCT Filed: Feb. 26, 2024
(86) PCT No.: PCT/KR2024/095445

§ 371 Date: May 9, 2024

(30) Foreign Application Priority Data
Feb. 28, 2023 (KR) 10-2023-0027010
Jan. 29, 2024 (KR) 10-2024-0012897

(51) Int. Cl. C12Q 1/689; A61K 35/00; A61K 35/741

(52) U.S. Cl. CPC C12Q 1/689; A61K 35/741; A61K 2035/11; C12Q 2600/156

(57) ABSTRACT

A personalized treatment means which can maximize therapeutic effects is disclosed. The personalized treatment means predicts the therapeutic efficacy of biotherapeutics, and provides, as a personalized treatment option, biologicals containing bacteria having no competitive exclusion relationship according to the distribution of strains or phylogroups of therapeutic bacteria in the gut sample of a patient.

### CROSS REFERENCE TO RELATED APPLICATIONS

This application is a National Stage of International Application No. PCT/KR2024/095445 filed Feb. 26, 2024, claiming priority based on Korean Patent Application No. 10-2023-0027010 filed Feb. 28, 2023 and Korean Patent Application No. 10-2024-0012897 filed Jan. 29, 2024.

### INCORPORATION BY REFERENCE OF SEQUENCE LISTING

The instant application contains a Sequence Listing which has been filed electronically in xml format and is hereby incorporated by reference in its entirety. Said xml copy, created on May 9, 2024, is named Sequence Listing US-ETB-P2410.xml and is 56.0 KB in size.

### TECHNICAL FIELD

The present invention relates to a method for predicting a therapeutic response of biotherapeutics and a treatment method of various diseases including a metabolic disorder using the predicted result, and more specifically, to providing personalized medicine by selecting, as a treatment option, biologicals containing bacteria having no competitive exclusion relationship according to the distribution of phylogroups of therapeutic bacteria in a sample of a patient.

### BACKGROUND ART

Obesity is a serious disease that has no effective treatments and is on the rise worldwide.

Unlike other diseases, obesity is characterized by involving related diseases such as metabolic syndrome, hypertension, diabetes mellitus, hyperlipidemia, arteriosclerosis, ischemic heart disease, fatty liver, and gallstone. Metabolic syndrome is a cluster of conditions in which abdominal obesity, impaired glucose tolerance, hypertension, and dyslipidemia occur together.

Antiobesity agents commercially available up to date are antiobesity agents that depend on chemicals and are largely divided into antiobesity agents of an appetite suppressant class or antiobesity agents of a lipid digestion inhibitor class. However, since antiobesity agents of an appetite suppressant class are substances that act on the central nervous system, most of them are being withdrawn from the market due to fatal problems that cause serious adverse effects when used for a long period of time. Meanwhile, orlistat (Xenical® and Alli® of Roche), the only drug that has successfully entered the market after clinical trials among antiobesity agents of a lipid digestion inhibitor class, has been reported to cause diarrhea and steatorrhea, and severer liver injury occurs when the drug is used for a long period of time, so that the U.S. FDA has been reviewing the safety of orlistat. As such, most of the currently marketed antiobesity agents have serious adverse effects, and antiobesity agents using enterobacteria are attracting attention as a promising treatment means in treating obesity and related disorders.

The term “pharmabiotics” is a compound word of pharmaceuticals and probiotics, and is defined as bacteria having a proven medical efficacy for health or disease or a metabolite produced by bacteria (Hill, 2010). In order for a pharmabiotics product to be approved as a drug by regulatory authorities such as the European Medicines Agency and the U.S. Food and Drug Administration, it must demonstrate a continuous and objective physiological and medical effect.

The efficacy of the pharmabiotics product may have big difference between individuals due to the significant inter-individual microbiome variability mediated by various factors such as age, health conditions, diet, whether to use antibiotics, and consumption of health functional foods of a subject (patient). Therefore, there is an urgent need to develop a technology that can select pharmabiotics suitable for each individual.

For example, an Akkermansia muciniphila strain, which is recognized as the next-generation microbiome, is attracting attention as a candidate for first-in-class drugs for obesity, metabolic syndrome, type 2 diabetes, and non-alcoholic fatty liver. Compared to its importance, such an Akkermansia strain has not been clearly identified for specific mechanisms. This is because most of the strain influence evaluation studies are being conducted on the standard strain, the Akkermansia muciniphila BAA-835 single strain. There are various Akkermansia strains in the intestine, but only the identification method at the whole genome level is used to detect this, so that there is a limitation that an accurate study is impossible.

### DISCLOSURE OF INVENTION

### Technical Problem

The present inventors have discovered for the first time that probiotic strains exist in a predominant form of a single strain or phylogroup in the intestine rather than in a complex form in which various strains or phylogroups are mixed and exhibit a competitive exclusion relationship with respect to other strains or phylogroups, thereby completing the present invention.

One object of the present invention is to provide a method for predicting a therapeutic response of a patient to biotherapeutics by using competitive superiority, settlement inhibition, and a competitive exclusion relationship between enterobacteria for gastrointestinal cells.

Another object of the present invention is to provide a method for predicting a therapeutic response of a patient to biotherapeutics by separating bacterial genes from a fecal sample that can be easily obtained and analyzing the genes with qPCR.

Further another object of the present invention is to provide a method for predicting a therapeutic response of a patient to biotherapeutics through a phylogroup-specific identification region in the 16S rRNA gene of bacteria.

Still another object of the present invention is to provide a marker composition for predicting a therapeutic response of a patient with a metabolic disorder to biotherapeutics including pharmabiotic bacteria.

Still yet another object of the present invention is to provide a method for treating various diseases such as metabolic disorders, the method providing biotherapeutics as a customized treatment option, wherein the biotherapeutics is capable of maximizing therapeutic effects by using competitive superiority, settlement inhibition, or a competitive exclusion relationship between enterobacteria.

However, the technical problems to be solved by the present invention are not limited to the above-mentioned problems, and other problems not mentioned will be clearly understood by those skilled in the art from the following description.

### Solution to Problem

In order to achieve the above-described objects, an aspect of the present invention relates to a method for predicting a therapeutic response of a patient to biotherapeutics, the method including:
(a) confirming the distribution of target bacteria for each strain or phylogroup through gut microbiota analysis of the patient;
(b) identifying whether there is a competitive exclusion relationship between a strain or phylogroup identified as a gut-dominant species of the patient in the previous step and a target strain or phylogroup; and
(c) determining that when the strain or phylogroup identified as the gut-dominant species and the strain or phylogroup of the target bacteria are identified to have a competitive exclusion relationship in the previous step, the therapeutic response of the patient to the biotherapeutics including the corresponding strain or phylogroup is low.

The gut microbiota analysis may include performing quantitative PCR (qPCR) on DNA extracted from a fecal sample of a patient using a primer pair or probe specific to the sodium ion-translocating decarboxylase subunit beta gene of bacteria of a specific strain or phylogroup.

In addition, the gut microbiota analysis may include performing identification and distribution confirmation of phylogroup on DNA extracted from a fecal sample of a patient by using a phylogroup-specific gene identification region specific to 16S rRNA gene of bacteria of a specific strain or phylogroup.

The identifying of the presence or absence of the competitive exclusion relationship may include treating the target strain or phylogroup with the culture supernatant of the strain or phylogroup identified as the gut-dominant species of the patient to determine whether the growth of the target strain or phylogroup is inhibited.

Another aspect of the present invention relates to a method for predicting a therapeutic response of a patient to biotherapeutics, the method including:
(a) confirming the distribution of Akkermansia sp. bacteria to be used as biotherapeutics for each strain or phylogroup through gut microbiota analysis of the patient; (b) identifying whether there is a competitive exclusion relationship between the Akkermansia sp. phylogroup identified as a gut-dominant species in the previous step and the target Akkermansia sp. strain or phylogroup; and
(c) determining that when the Akkermansia sp. phylogroup identified as the gut-dominant species and the target Akkermansia sp. bacteria are identified to have a competitive exclusion relationship in the previous step, the therapeutic response of the patient to the biotherapeutics including the corresponding Akkermansia sp. strain or phylogroup is low.

Still another aspect of the present invention relates to a method for treating patients with various diseases including a metabolic disorder, the method including:
(a) confirming the distribution of target bacteria for each strain or phylogroup through gut microbiota analysis of the patient;
(b) identifying whether there is a competitive exclusion relationship between a strain or phylogroup identified as a gut-dominant species of the patient in the previous step and a target strain or phylogroup; and
(c) when the strain or phylogroup identified as the dominant species and the strain or phylogroup of the target bacteria are identified to have no competitive exclusion relationship in the previous step, selecting, as a treatment option, biotherapeutics including the corresponding strain or phylogroup to administer the biotherapeutics to the patient.

### Advantageous Effects of Invention

According to the present invention, it is possible to specifically detect each of four phylogroups of AmIa, AmIb, AmII, and AmIV belonging to the Akkermansia strain forming a gut microbiome. The present invention not only can accurately and clearly detect the Akkermansia strain having various distribution depending on various diseases, and specifically and accurately detect each of the four strains belonging to the Akkermansia strain. Therefore, it is possible to analyze a prevalence of a specific disease and the corresponding strain, and a customized diet or drug containing the target strain can be provided on the basis of the analyzed prevalence.

No matter how many pharmabiotic bacteria are consumed, the pharmabiotic bacteria cannot show efficacy if the pharmabiotic bacteria are not able to survive and reach the intestine or are not dominant after settling in the intestine. According to the present invention, since through gut microbiota analysis of each patient, pharmabiotics that can easily settle in the intestine of the corresponding patient can be selected and administered, the treatment effect of pharmabiotics can be maximized.

The present invention provides a method for verifying personalized probiotics, prebiotics, food, health functional food, and medicine on the basis of the gut microbiota of a patient, thereby providing an effective analysis method for screening an effective strain capable of treating metabolic disorders and the like in a personalized manner.

### BRIEF DESCRIPTION OF DRAWINGS

FIG. 1 is a view showing three phylogroup-identification regions capable of specifically identifying the Akkermansia phylogroup (AmI, AmII, and AmIV) in the 16S rRNA gene sequence of Akkermansia. In FIG. 1, the sequence of AmI is SEQ ID NO: 21, the sequence of AMII is SEQ ID NO: 22, and the sequence of AmIV is SEQ ID NO: 23.

FIG. 2a is a phylogram of 92 types of Akkermansia on the basis of the 16S rRNA gene sequence, and FIG. 2b is a graph showing average nucleotide identity relative to the whole genome of 92 types of Akkermansia strains.

FIG. 3a shows the phylogenetic classification of 92 types of Akkermansia based on the sodium ion-translocating decarboxylase subunit beta gene sequence, which is the marker gene, and FIG. 3b is a graph showing the genetic differences between intra-phylogroups or inter-phylogroups of the marker gene.

FIG. 4a is a circular phylogram of 22 amplicon sequence variants (ASVs) belonging to the Akkermansia sp. identified by the 16S rRNA sequence of 92 human-associated Akkermansia genome and the 16S amplicon sequencing data of 890 Koreans, and FIG. 4b is the distribution diagram showing the distribution of Akkermansia for each phylogroup in the intestines of 890 Koreans.

FIG. 5a is a circular phylogram showing the presence or absence of Akkermansia in seven countries including Korea and distribution patterns for each phylogroup, and FIG. 5b is the distribution diagram showing the distribution of Akkermansia phylogroups in seven countries including Korea.

FIG. 6 is a diagram showing the results of evaluating the analytical performance of the Akkermansia phylogroup-specific primers (AmIa, AmIb, and AmII) according to an embodiment of the present invention.

FIGS. 7a-7c are diagram showing retention of settlement and changes in fecal phase levels after mono-administration of a representative strain of each Akkermansia phylogroup to germ-free mice.

FIGS. 8a and 8b are graph showing that there is a competitive exclusion relationship when various Akkermansia phylogroup strains are simultaneously administered to germ-free mice. FIG. 8a shows the results of confirming the settlement in the gut of mice when various Akkermansia phylogroups (BAA-835: AmIa, EB-AMDK19: AmIb, and EB-AMDK39: AmII) were simultaneously administered. FIG. 8b shows the results of confirming the settlement in the gut of mice when two types of Akkermansia phylogroups (EB-AMDK19: AmIb, and EB-AMDK39: AmII) were simultaneously administered.

FIG. 9a is a photograph showing the electrophoresis results of PCR using strain-specific and phylogroup-specific primers, and FIG. 9b shows melting curve plots of qPCR using strain-specific and phylogroup-specific primers.

FIGS. 10a and 10b are graph showing changes in the gut Akkermansia phylogroups when the AmI and AmII phylogroups are cross-administered to germ-free mice.

### MODE FOR THE INVENTION

Unless otherwise defined herein, the scientific and technical terms used herein will have meanings commonly understood by those skilled in the art.

In the present specification, when it is described that one part “comprises” or “includes” some components, it is not meant as the exclusion of the other components but to implies the further inclusion of the other components, unless explicitly stated to the contrary.

As used herein, the terms “patient” and “subject” may be used interchangeably and may refer to a human or non-human animal. These terms include mammals such as humans, non-human primates, livestock (e.g., cattle, pigs, sheep, goats, and poultry), companion animals (e.g., dogs, cats, horses, and oryctolagus) and rodents (e.g., guinea pigs, hamsters, and mice).

As used herein, the terms such as “treat” and “treatment” mean that symptoms are temporarily or permanently relieved, the cause of the symptoms is removed, or the development of symptoms of a disease or condition is combated or delayed.

As used herein, the terms “biotherapeutics” and “biomedicine” are used interchangeably, and refer to a drug including bacteria (probiotics) which is effective in preventing, treating, or curing a disease or disorder. In an embodiment of the present invention, the “biotherapeutics” consists of or includes anaerobic bacteria or obligate anaerobic bacteria.

As used herein, the term “target bacteria” refers to therapeutic bacteria (probiotics) to be used as biotherapeutics in a specific subject or patient.

As used herein, the term “gut microbiota analysis” refers to a test for analyzing the composition and/or distribution of various bacteria present in the gut through gene analysis of bacteria or microbiota discharged through feces.

As used herein, the term “primer” refers to a short nucleic acid sequence having a short free 3′ hydroxyl group, which can form a base pair acting with a complementary template and is a starting point for copying the template. The primer can initiate DNA synthesis in the presence of a reagent for polymerization (i.e., DNA polymerase or reverse transcriptase) and four different nucleoside triphosphates in an appropriate buffer at an appropriate temperature.

As used herein, the term “sequence homology,” “percent homology,” or “percent identity” refers to a degree to which sequences are identical based on nucleotide-by-nucleotide over a comparison window.

As used herein, the term “metabolic disorder” refers to obesity, metabolic syndrome, insulin-deficiency or insulin-resistance related disorders, diabetes mellitus (e.g., type 2 diabetes), glucose intolerance, abnormal lipid metabolism, atherosclerosis, hypertension, cardiac pathology, stroke, non-alcoholic fatty liver disease, hyperglycemia, fatty liver, dyslipidemia, dysfunction of the immune system associated with overweight and obesity, cardiovascular diseases, high cholesterol, elevated triglyceride, asthma, sleep apnea, osteoarthritis, neuro-degeneration, gallbladder disease, syndrome X, inflammatory disease, immune disease, atherogenic dyslipidemia, and cancer. In another embodiment, said metabolic disorder is an overweight and/or obesity related metabolic disorder, i.e., a metabolic disorder that may be associated to or caused by overweight and/or obesity. Examples of overweight and/or obesity related metabolic disorder include, but are not limited to, metabolic syndrome, insulin-deficiency or insulin-resistance related disorders, diabetes mellitus (e.g., type 2 diabetes), glucose intolerance, abnormal lipid metabolism, atherosclerosis, hypertension, cardiac pathology, stroke, non-alcoholic fatty liver disease, hyperglycemia, fatty liver, dyslipidemia, dysfunction of the immune system associated with overweight and obesity, cardiovascular diseases, high cholesterol, elevated triglycerides, asthma, sleep apnea, osteoarthritis, neuro-degeneration, gallbladder disease, syndrome X, inflammatory and immune disorders, atherogenic dyslipidemia, and cancer.

As used herein, the term “gut dominant species” refers to a bacteria species(s) or a phylogroup(s) identified as dominant species in the intestines of the patient.

test for analyzing the composition and/or distribution of various bacteria present in the gut through gene analysis of bacteria or microbiota discharged through feces.

An aspect of the present invention relates to a method for predicting a therapeutic response of a patient to biotherapeutics, the method including:
(a) confirming the distribution of target bacteria for each strain or phylogroup through gut microbiota analysis of the patient;
(b) identifying whether there is a competitive exclusion relationship between a strain or phylogroup identified as a gut-dominant species of the patient in the previous step and a target strain or phylogroup; and
(c) determining that when the strain or phylogroup identified as the gut-dominant species and the strain or phylogroup of the target bacteria are identified to have a competitive exclusion relationship in the previous step, the therapeutic response of the patient to the biotherapeutics including the corresponding strain or phylogroup is low.

In one aspect of the present invention, DNA is analyzed from intestinal samples in the gut microbiota analysis, the intestinal samples are fecal samples, and DNA is extracted from the intestinal samples before gene analysis. A method for extracting DNA of probiotics in fecal samples is not particularly limited, uses a combination of mechanical disruption, such as high speed bead beating extraction, chemical lysis and a final purification step, by using silica membrane column such as those included in a commercially available DNA extraction kit.

A method for confirming the distribution for each strain or phylogroup in intestinal bacteria of a patient may be performed by using a classical and appropriate method known in the art to which the present invention pertains. In general, it is carried out by gene quantification of bacteria that measures the amount or relative abundance of a specific nucleic acid sequence in a sample.

The gut microbiota analysis may be performed by quantitative PCR (qPCR) on DNA extracted from a fecal sample of a patient using a specific primer pair or probe of bacteria of a specific strain or phylogroup.

In a preferred embodiment, the quantification of the sodium ion-translocating decarboxylase subunit beta gene of the target bacteria may be carried out using a strain- or phylogroup-specific primers in Table 1 below or one or more oligonucleotide molecules of a sequence having at least 75% sequence homology thereto.

TABLE 1

| Phylo- | | | Akkermansia phylogroup- | Amplicon |
|---|---|---|---|---|
| group | SEQ ID NO. | Direction | specific primer | size (bp) |
| AmIa | SEQ ID NO: 1 | Forward | CGCTTCAGCAGGCTC | 253 |
| | SEQ ID NO: 2 | Reverse | GGTTCATTGCCGTTGTC | |
| AmIb | SEQ ID NO: 3 | Forward | ATGTGGCTCCTCTGCA | 358 |
| | SEQ ID NO: 4 | Reverse | AGGTCCACGGGAACA | |
| AmII | SEQ ID NO: 5 | Forward | TTGGCGCATTAATTGC | 466 |
| | SEQ ID NO: 6 | Reverse | TCCGTGTCAAAGTAGTTGACT | |
| AmIV | SEQ ID NO: 7 | Forward | GACTTCTCCAATGTCAGCG | 642 |
| | SEQ ID NO: 8 | Reverse | TCCCTTGCTGACCTGC | |

Preferably, oligonucleotide sequences having at least 75% sequence homology described herein have at least 80%, at least 85%, at least 90%, at least 95%, more preferably 96%, 97%, 98%, 99% or 100% sequence homology with the corresponding sequence (e.g., SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8, respectively); particularly preferred are nucleotide molecules having 100% sequence homology. In addition, these oligonucleotide sequences having at least 75% sequence homology may have the same number of nucleotides.

For example, when the target bacteria are Akkermansia, the target bacteria may be analyzed by qPCR using an AmIa-specific primer having at least 75% sequence homology to the sequence of SEQ ID NO: 1 or SEQ ID NO: 2; an AmIb-specific primer having at least 75% sequence homology to the sequence of SEQ ID NO: 3 or SEQ ID NO: 4; an AmII-specific primer having at least 75% sequence homology to the sequence of SEQ ID NO: 5 or SEQ ID NO: 6; or an AmIV-specific primer having at least 75% sequence homology to the sequence of SEQ ID NO: 7 or SEQ ID NO: 8.

Alternatively, the whole DNA extracted from human feces, or mucosal or tissue samples may be analyzed by 16S rRNA gene sequencing, such as Sanger, 454 pyrosequencing, MiSeq or HiSeq technology.

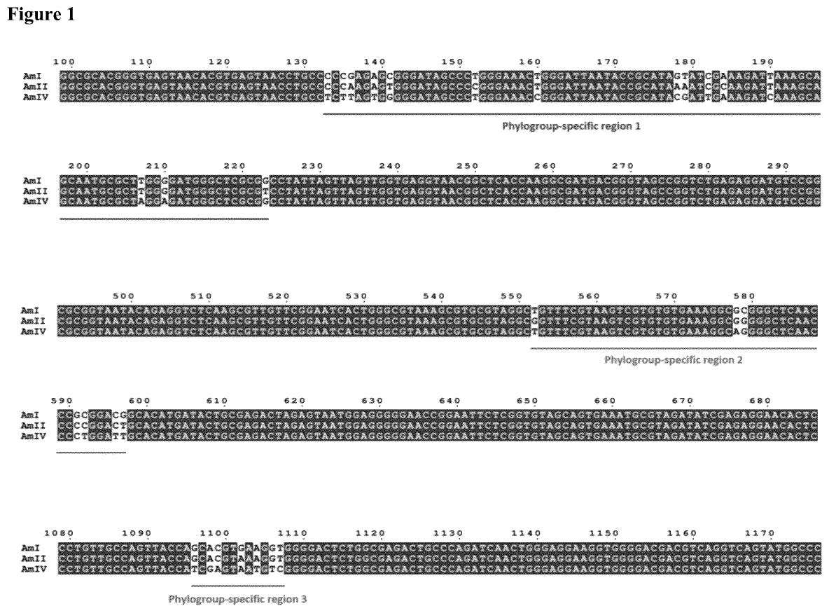

Phylogroup discrimination based on the 16S rRNA gene of the target bacteria which is analyzed via sequencing may be performed using the strain- or phylogroup-specific gene sequences of FIG. 1 and Table 2 below. That is, the Akkermansia phylogroups may be discriminated through phylogroup identification region 1, phylogroup identification region 2, and phylogroup identification region 3.

TABLE 2

| | | | Gene sequence of phylogroup- |
|---|---|---|---|
| 16S rRNA phylogroup | Phylo- | SEQ ID | specific identification region |
| identification region | group | NO. | (5′→3′) |
| Phylogroup | AmI | SEQ ID | CCCGAGAGCGGGATAGCCCTGGGAAACTG |
| identification | | NO: 9 | GGATTAATACCGCATAGTATCGAAAGATTA |
| region 1 (91 bp) | | | AAGCAGCAATGCGCTTGGGGATGGGCTCGC |
| | | | GG |
| | AmII | SEQ ID | CCCAAGAGTGGGATAGCCCCGGGAAACTG |
| | | NO: 10 | GGATTAATACCGCATAAAATCGCAAGATTA |
| | | | AAGCAGCAATGCGCTTGGGGATGGGCTCGC |
| | | | GT |
| | AmIV | SEQ ID | TCTTAGTGGGGGATAGCCCTGGGAAACCG |
| | | NO: 11 | GGATTAATACCGCATACGATTGAAAGATCA |
| | | | AAGCAGCAATGCGCTAGGAGATGGGCTCGC |
| | | | GG |
| Phylogroup | AmI | SEQ ID | TGTTTCGTAAGTCGTGTGTGAAAGGCGCGG |
| identification | | NO: 12 | GCTCAACCCGCGGACG |
| region 2 (46 bp) | AmII | SEQ ID | GGTTTCGTAAGTCGTGTGTGAAAGGCGGGG |
| | | NO: 13 | GCTCAACCCCCGGACT |
| | AmIV | SEQ ID | TGTTTCGTAAGTCGTGTGTGAAAGGCAGGG |
| | | NO: 14 | GCTCAACCCCTGGATT |
| Phylogroup | AmI | SEQ ID | GCACGTGAAGGT |
| identification | | NO: 15 | |
| region 3 (12 bp) | AmII | SEQ ID | GCACGTAAAGGT |
| | | NO: 16 | |
| | AmIV | SEQ ID | TCGAGTAATGTC |
| | | NO: 17 | |

Preferably, oligonucleotide sequences having at least 7500 sequence homology to the sequences of the phylogroup-specific identification region described herein have at least 8000, at least 85%, at least 90%, at least 95%, more preferably 96%, 97%, 98%, 99% or 100% sequence homology with the gene sequence of the corresponding phylogroup-specific identification region (e.g., phylogroup identification region 1, phylogroup identification region 2, phylogroup identification region 3, respectively); particularly preferred are nucleotide molecules having 100% sequence homology. In addition, these oligonucleotide sequences having at least 750% sequence homology may have the same number of nucleotides.

For example, when the target bacteria are Akkermansia, the Akkermansia phylogroup-specific identification region may be: phylogroup identification region 1 having at least 7500 sequence homology to the gene sequence of the AmI-specific identification region of SEQ TD NO: 9, the gene sequence of the AmII-specific identification region of SEQ ID NO: 10, or the gene sequence of the AmIV-specific identification region of SEQ ID NO: 11;
phylogroup identification region 2 having at least 75% sequence homology to the gene sequence of the AmI-specific identification region of SEQ ID NO: 12, the gene sequence of the AmII-specific identification region of SEQ ID NO: 13, or the gene sequence of the AmIV-specific identification region of SEQ ID NO: 14; or
phylogroup identification region 3 having at least 75% sequence homology to the gene sequence of the AmI-specific identification region of SEQ ID NO: 15, the gene sequence of the AmII-specific identification region of SEQ ID NO: 16, or the gene sequence of the AmIV-specific identification region of SEQ ID NO: 17.

The identifying of the presence or absence of the competitive exclusion relationship may be performed by a method for treating the target strain or the phylogroup with the culture supernatant of the Akkermansia phylogroup identified as the gut-dominant species of the subject to determine whether the growth of the target strain or the phylogroup is inhibited.

For example, a cell-free culture supernatant is obtained through centrifugation after 24-hours culturing of the Akkermansia strain belonging to the same phylogroup as Akkermansia identified as a gut-dominant species of the subject. The obtained culture supernatant is adjusted to neutral pH and is then added at a ratio of 20% (v/v) in culturing the target strain to be administered. By checking the culture after 24 hours, whether there is a competitive exclusion relationship is identified.

Preferably, the target bacteria in the present invention are Akkermansia. The Akkermansia has phylogroups AmIa, AmIb, AmII, and AmIV. Among the Akkermansia phylogroups, the phylogroups AmI and AmII have a competitive exclusion relationship, and the phylogroups AmIa and AmIb have a competitive exclusion relationship. Phylogroups AmIa and AmIb are inhibited by phylogroups AmII and AmIV, and phylogroup AmII inhibits phylogroups AmIa and AmIb, but is not inhibited by phylogroups AmIa and AmIb. Phylogroup AmIV inhibits the growth of phylogroup AmIa and AmIb and phylogroup AmII, but is not inhibited by phylogroup AmIa and AmIb and phylogroup AmII.

The method of the present invention may be used for screening or treating a patient for treating a metabolic disorder, but is not necessarily limited to a metabolic disorder. The method of the present invention may be used to maximize the therapeutic effect when treating various diseases such as inflammatory diseases, brain diseases, atopic diseases, and cancer by using pharmabiotics or postbiotics. In the present invention, the patient with a metabolic disorder may be a patient with metabolic syndrome, insulin-deficiency or insulin-resistance related disorders, diabetes mellitus, glucose intolerance, abnormal lipid metabolism, atherosclerosis, hypertension, pre-eclampsia, stroke, non-alcoholic fatty liver disease, hyperglycemia, hepatic steatosis, dyslipidemia, Crohn's disease, ulcerative colitis, irritable bowel syndrome, cardiovascular diseases, cerebrovascular diseases, peripheral vascular diseases, high cholesterol, elevated triglyceride, asthma, atopic dermatitis, sleep apnea, osteoarthritis, neurodegeneration, gallbladder diseases, or atherogenic dyslipidemia, but is not limited thereto.

Another aspect of the present invention relates to a marker composition for predicting a therapeutic response of a patient to biotherapeutics including Akkermansia sp. bacteria, wherein the marker composition may include an AmIa-specific primer having at least 75% sequence homology to the sequence of SEQ ID NO: 1 or SEQ ID NO: 2; an AmIb-specific primer having at least 75% sequence homology to the sequence of SEQ ID NO: 3 or SEQ ID NO: 4; an AmII-specific primer having at least 75% sequence homology to the sequence of SEQ ID NO: 5 or SEQ ID NO: 6; or an AmIV-specific primer having at least 75% sequence homology to the sequence of SEQ ID NO: 7 or SEQ ID NO: 8.

Further aspects of the present invention relate to nucleic acid molecules having a sequence selected from the group consisting of SEQ ID NO: 1 to SEQ ID NO: 8 or an oligonucleotide sequence that is at least 75% identical to the sequence. Preferably, the oligonucleotide sequence that is at least 75% identical has at least 80%, at least 85%, at least 90%, at least 95%, more preferably, 96%, 97%, 98%, 99% or 100% sequence homology with SEQ ID NO: 1, SEQ ID NO: 2, SEQ ID NO: 3, and/or SEQ ID NO: 4.

Still another aspect of the present invention relates to a marker composition for predicting a therapeutic response of a patient to biotherapeutics including Akkermansia sp. bacteria, wherein the marker composition is a specific phylogroup identification region capable of identifying the Akkermansia phylogroup of the patient, wherein the identification region may include phylogroup identification region 1 having at least 75% sequence homology to the gene sequence of SEQ ID NO: 9 to SEQ ID NO: 11; phylogroup identification region 2 having at least 75% sequence homology to the sequence of SEQ ID NO: 12 to SEQ ID NO: 14; and phylogroup identification region 3 having at least 75% sequence homology to the gene sequence of SEQ ID NO: 15 to SEQ ID NO: 17. Moreover, the Akkermansia phylogroup identification region may include a sequence selected from the group consisting of SEQ ID NO: 9 to SEQ ID NO: 17 or an oligonucleotide sequence at least 75% identical to the sequence. Preferably, the oligonucleotide sequence that is at least 75% identical has at least 80%, at least 85%, at least 90%, at least 95%, more preferably, 96%, 97%, 98%, 99% or 100% sequence homology with phylogroup identification region 1, phylogroup identification region 2, and phylogroup identification region 3.

Yet another aspect of the present invention includes:
(a) confirming the distribution of Akkermansia sp. bacteria to be used as biotherapeutics for each strain or phylogroup through gut microbiota analysis of a patient based on the 16S rRNA gene;
(b) identifying whether there is a competitive exclusion relationship between the Akkermansia sp. strain or phylogroup identified as a gut-dominant species in the previous step and the target Akkermansia sp. strain or phylogroup; and
(c) determining that when the Akkermansia sp. strain or phylogroup identified as the gut-dominant species and the target Akkermansia sp. bacteria are identified to have a competitive exclusion relationship in the previous step, the therapeutic response of the patient to the biotherapeutics including the corresponding Akkermansia sp. strain or phylogroup is low.

The patient may be a patient with a metabolic disorder.

Still yet another aspect of the present invention relates to a method for treating a patient with a metabolic disorder, the method including:
(a) confirming the distribution of target bacteria for each strain or phylogroup through gut microbiota analysis of the patient;
(b) identifying whether there is a competitive exclusion relationship between a strain or phylogroup identified as a gut-dominant species of the patient in the previous step and a target strain or phylogroup; and
(c) when the strain or phylogroup identified as the dominant species and the strain or phylogroup of the target bacteria are identified to have no competitive exclusion relationship in the previous step, selecting, as a treatment option, biotherapeutics including the corresponding target strain or phylogroup to administer the biotherapeutics to the patient.

The metabolic disorder may be selected from the group consisting of metabolic syndrome, insulin-deficiency or insulin-resistance related disorders, diabetes mellitus, glucose intolerance, abnormal lipid metabolism, atherosclerosis, hypertension, pre-eclampsia, stroke, non-alcoholic fatty liver disease, hyperglycemia, hepatic steatosis, dyslipidemia, inflammatory diseases including Crohn's disease, ulcerative colitis, and irritable bowel syndrome, cardiovascular diseases, cerebrovascular diseases, peripheral vascular diseases, high cholesterol, elevated triglyceride, asthma, atopic dermatitis, sleep apnea, osteoarthritis, neurodegeneration, gallbladder diseases, and atherogenic dyslipidemia, but is not necessarily limited thereto.

Hereinafter, the present invention will be described in more detail with reference to Examples. The present examples are for describing the present invention in more detail, and the scope of the present invention is not limited by these examples.

### EXAMPLES

### Example 1

### Example 1.1: Differences in Genetic Characteristics Between Akkermansia Phylogroups

The present inventors collected 44 Akkermansia isolates from 19 Koreans and the whole genome was sequenced by PacBio platform. In addition, to investigate the genome diversity of Akkermansia, 48 complete human-associated Akkermansia genomes were downloaded from the NCBI Reference Sequence project (RefSeq) database (www.ncbi.nlm.nih.gov/refseq/).

Referring to Table 3, a total of 92 types of complete Akkermansia genomes showed various genome sizes ranging from 2.66 Mbp to 3.30 Mbp (average 2.87 Mbp). This indicates that the sizes of the genomes of various Akkermansia differ significantly by 0.64 Mbp. Almost all of the genome sizes of Akkermansia isolated from humans were larger than those of ATCC BAA-835. The number of genes encoding protein in the 92 available protein genomes varied from 2122 to 2728. In addition, the 92 Akkermansia genomes had the same number of rRNA genes and the rRNA genes including 5 S, 16S, and 23 S were identical to each other at 3, 3, 3.

TABLE 3

| Strain | Assembly_level | Seq_category | Total_size | GC (%) | Genes | CDS | Coding | RNA | rRNA | tRNA | cRNA |
|---|---|---|---|---|---|---|---|---|---|---|---|
| AK32 | Complete | 1 Chromosome | 3004919 | 55.3 | 2611 | 2544 | 2493 | 67 | 3, | 55 | 3 |
| | | | | | | | | | 3, 3 | | |
| Akk1756 | Complete | 1 Chromosome | 2942163 | 55.5 | 2505 | 2439 | 2417 | 66 | 3, | 54 | 3 |
| | | | | | | | | | 3, 3 | | |
| JCM30893 | Complete | 1 Chromosome | 2878261 | 55.6 | 2435 | 2369 | 2363 | 66 | 3, | 54 | 3 |
| | | | | | | | | | 3, 3 | | |
| CBA5201 | Complete | 1 Chromosome | 2860407 | 55.3 | 2433 | 2367 | 2351 | 66 | 3, | 54 | 3 |
| | | | | | | | | | 3, 3 | | |
| Akk2090 | Complete | 1 Chromosome | 2803336 | 55.3 | 2395 | 2329 | 2321 | 66 | 3, | 54 | 3 |
| | | | | | | | | | 3, 3 | | |
| Akk2030 | Complete | 1 Chromosome | 2803334 | 55.3 | 2392 | 2326 | 2312 | 66 | 3, | 54 | 3 |
| | | | | | | | | | 3, 3 | | |
| Akk1990 | Complete | 1 Chromosome | 2803293 | 55.3 | 2389 | 2323 | 2312 | 66 | 3, | 54 | 3 |
| | | | | | | | | | 3, 3 | | |
| EB- | Complete | 1 Chromosome | 2803530 | 55.7 | 2384 | 2318 | 2304 | 66 | 3, | 54 | 3 |
| AMDK26 | | | | | | | | | 3, 3 | | |
| Akk1863 | Complete | 1 Chromosome | 3309705 | 57.8 | 2800 | 2735 | 2728 | 65 | 3, | 53 | 3 |
| | | | | | | | | | 3, 3 | | |
| EB- | Complete | 1 Chromosome | 3208499 | 57.78 | 2813 | 2748 | 2692 | 65 | 3, | 53 | 3 |
| AMDK43 | | | | | | | | | 3, 3 | | |
| AkkB40 | Complete | 1 Chromosome | 3163055 | 57.6 | 2639 | 2574 | 2560 | 65 | 3, | 53 | 3 |
| | | | | | | | | | 3, 3 | | |
| Akk2680 | Complete | 1 Chromosome | 3125827 | 58 | 2632 | 2567 | 2556 | 65 | 3, | 53 | 3 |
| | | | | | | | | | 3, 3 | | |
| Akk2000 | Complete | 1 Chromosome | 3119604 | 58 | 2637 | 2572 | 2555 | 65 | 3, | 53 | 3 |
| | | | | | | | | | 3, 3 | | |
| EB- | Complete | 1 Chromosome | 3156483 | 57.8 | 2630 | 2565 | 2552 | 65 | 3, | 53 | 3 |
| AMDK40 | | | | | | | | | 3, 3 | | |
| Akk2190 | Complete | 1 Chromosome | 3119586 | 58 | 2621 | 2556 | 2548 | 65 | 3, | 53 | 3 |
| | | | | | | | | | 3, 3 | | |
| Akk1476 | Complete | 1 Chromosome | 3119572 | 58 | 2633 | 2568 | 2548 | 65 | 3, | 53 | 3 |
| | | | | | | | | | 3, 3 | | |
| EB- | Complete | 1 Chromosome | 3156561 | 57.8 | 2626 | 2561 | 2547 | 65 | 3, | 53 | 3 |
| AMDK39 | | | | | | | | | 3, 3 | | |
| EB- | Complete | 1 Chromosome | 3156416 | 57.8 | 2616 | 2551 | 2539 | 65 | 3, | 53 | 3 |
| AMDK41 | | | | | | | | | 3, 3 | | |
| Akk2196 | Complete | 1 Chromosome | 3119569 | 58 | 2614 | 2549 | 2530 | 65 | 3, | 53 | 3 |
| | | | | | | | | | 3, 3 | | |
| EB- | Complete | 1 Chromosome | 2844797 | 55.9 | 2438 | 2373 | 2365 | 65 | 3, | 53 | 3 |
| AMDK47 | | | | | | | | | 3, 3 | | |
| EB- | Complete | 1 Chromosome | 2844777 | 55.9 | 2426 | 2361 | 2354 | 65 | 3, | 53 | 3 |
| AMDK48 | | | | | | | | | 3, 3 | | |
| EB- | Complete | 1 Chromosome | 2844808 | 55.9 | 2425 | 2360 | 2352 | 65 | 3, | 53 | 3 |
| AMDK49 | | | | | | | | | 3, 3 | | |
| EB- | Complete | 1 Chromosome | 2844782 | 55.9 | 2423 | 2358 | 2350 | 65 | 3, | 53 | 3 |
| AMDK46 | | | | | | | | | 3, 3 | | |
| EB- | Complete | 1 Chromosome | 2836174 | 55.4 | 2399 | 2334 | 2316 | 65 | 3, | 53 | 3 |
| AMDK31 | | | | | | | | | 3, 3 | | |
| EB- | Complete | 1 Chromosome | 2836855 | 55.3 | 2401 | 2336 | 2313 | 65 | 3, | 53 | 3 |
| AMDK38 | | | | | | | | | 3, 3 | | |
| EB- | Complete | 1 Chromosome | 2836880 | 55.3 | 2380 | 2315 | 2298 | 65 | 3, | 53 | 3 |
| AMDK37 | | | | | | | | | 3, 3 | | |
| EB- | Complete | 1 Chromosome | 2782314 | 55.7 | 2400 | 2335 | 2297 | 65 | 3, | 53 | 3 |
| AMDK24 | | | | | | | | | 3, 3 | | |
| EB- | Complete | 1 Chromosome | 2824041 | 55.4 | 2396 | 2331 | 2295 | 65 | 3, | 53 | 3 |
| AMDK8 | | | | | | | | | 3, 3 | | |
| EB- | Complete | 1 Chromosome | 2810249 | 55.3 | 2374 | 2309 | 2291 | 65 | 3, | 53 | 3 |
| AMDK6 | | | | | | | | | 3, 3 | | |
| EB- | Complete | 1 Chromosome | 2782478 | 55.7 | 2359 | 2294 | 2283 | 65 | 3, | 53 | 3 |
| AMDK23 | | | | | | | | | 3, 3 | | |
| Akk0500b | Complete | 1 Chromosome | 2788976 | 55.4 | 2370 | 2305 | 2282 | 65 | 3, | 53 | 3 |
| | | | | | | | | | 3, 3 | | |
| EB- | Complete | 1 Chromosome | 2782334 | 55.7 | 2363 | 2298 | 2275 | 65 | 3, | 53 | 3 |
| AMDK25 | | | | | | | | | 3, 3 | | |
| EB- | Complete | 1 Chromosome | 2763834 | 55.2 | 2434 | 2369 | 2260 | 65 | 3, | 53 | 3 |
| AMDK10 | | | | | | | | | 3, 3 | | |
| EB- | Complete | 1 Chromosome | 2763965 | 55.3 | 2411 | 2346 | 2247 | 65 | 3, | 53 | 3 |
| AMDK13 | | | | | | | | | 3, 3 | | |
| EB- | Complete | 1 Chromosome | 2770146 | 55.3 | 2357 | 2292 | 2244 | 65 | 3, | 53 | 3 |
| AMDK17 | | | | | | | | | 3, 3 | | |
| EB- | Complete | 1 Chromosome | 2770098 | 55.3 | 2364 | 2299 | 2243 | 65 | 3, | 53 | 3 |
| AMDK15 | | | | | | | | | 3, 3 | | |
| EB- | Complete | 1 Chromosome | 2770124 | 55.3 | 2357 | 2292 | 2242 | 65 | 3, | 53 | 3 |
| AMDK18 | | | | | | | | | 3, 3 | | |
| EB- | Complete | 1 Chromosome | 2764188 | 55.3 | 2349 | 2284 | 2240 | 65 | 3, | 53 | 3 |
| AMDK14 | | | | | | | | | 3, 3 | | |
| EB- | Complete | 1 Chromosome | 2764211 | 55.3 | 2329 | 2264 | 2235 | 65 | 3, | 53 | 3 |
| AMDK2 | | | | | | | | | 3, 3 | | |
| EB- | Complete | 1 Chromosome | 2764297 | 55.3 | 2320 | 2255 | 2227 | 65 | 3, | 53 | 3 |
| AMDK12 | | | | | | | | | 3, 3 | | |
| EB- | Complete | 1 Chromosome | 2764311 | 55.3 | 2320 | 2255 | 2224 | 65 | 3, | 53 | 3 |
| AMDK11 | | | | | | | | | 3, 3 | | |
| EB- | Complete | 1 Chromosome | 2734231 | 55.4 | 2303 | 2238 | 2222 | 65 | 3, | 53 | 3 |
| AMDK27 | | | | | | | | | 3, 3 | | |
| EB- | Complete | 1 Chromosome | 2734254 | 55.4 | 2308 | 2243 | 2221 | 65 | 3, | 53 | 3 |
| AMDK28 | | | | | | | | | 3, 3 | | |
| Akk0490 | Complete | 1 Chromosome | 3208715 | 56.7 | 2673 | 2609 | 2591 | 64 | 3, | 52 | 3 |
| | | | | | | | | | 3, 3 | | |
| Akk0196 | Complete | 1 Chromosome | 3212887 | 56.7 | 2672 | 2608 | 2587 | 64 | 3, | 52 | 3 |
| | | | | | | | | | 3, 3 | | |
| Akk0496b | Complete | 1 Chromosome | 3208743 | 56.7 | 2663 | 2599 | 2582 | 64 | 3, | 52 | 3 |
| | | | | | | | | | 3, 3 | | |
| Akk0496a | Complete | 1 Chromosome | 3174614 | 56.8 | 2619 | 2555 | 2538 | 64 | 3, | 52 | 3 |
| | | | | | | | | | 3, 3 | | |
| Akk2750 | Complete | 1 Chromosome | 3174619 | 56.8 | 2615 | 2551 | 2535 | 64 | 3, | 52 | 3 |
| | | | | | | | | | 3, 3 | | |
| Akk0580 | Complete | 1 Chromosome | 3083850 | 58 | 2588 | 2524 | 2514 | 64 | 3, | 52 | 3 |
| | | | | | | | | | 3, 3 | | |
| Akk1570 | Complete | 1 Chromosome | 2965470 | 55.3 | 2560 | 2496 | 2478 | 64 | 3, | 52 | 3 |
| | | | | | | | | | 3, 3 | | |
| Akk1576 | Complete | 1 Chromosome | 2977681 | 55.3 | 2556 | 2492 | 2467 | 64 | 3, | 52 | 3 |
| | | | | | | | | | 3, 3 | | |
| Akk1573 | Complete | 1 Chromosome | 3049079 | 58.1 | 2541 | 2477 | 2464 | 64 | 3, | 52 | 3 |
| | | | | | | | | | 3, 3 | | |
| Akk0880 | Complete | 1 Chromosome | 2965460 | 55.3 | 2549 | 2485 | 2463 | 64 | 3, | 52 | 3 |
| | | | | | | | | | 3, 3 | | |
| Akk16115 | Complete | 1 Chromosome | 3002684 | 55.1 | 2541 | 2477 | 2454 | 64 | 3, | 52 | 3 |
| | | | | | | | | | 3, 3 | | |
| Akk16145 | Complete | 1 Chromosome | 3002718 | 55.1 | 2551 | 2487 | 2453 | 64 | 3, | 52 | 3 |
| | | | | | | | | | 3, 3 | | |
| Akk1610 | Complete | 1 Chromosome | 3002694 | 55.1 | 2544 | 2480 | 2451 | 64 | 3, | 52 | 3 |
| | | | | | | | | | 3, 3 | | |
| Akk1616 | Complete | 1 Chromosome | 3002740 | 55.1 | 2538 | 2474 | 2448 | 64 | 3, | 52 | 3 |
| | | | | | | | | | 3, 3 | | |
| Akk1613 | Complete | 1 Chromosome | 2990835 | 55.2 | 2530 | 2466 | 2434 | 64 | 3, | 52 | 3 |
| | | | | | | | | | 3, 3 | | |
| EB- | Complete | 1 Chromosome | 2888371 | 55.5 | 2463 | 2399 | 2388 | 64 | 3, | 52 | 3 |
| AMDK29 | | | | | | | | | 3, 3 | | |
| EB- | Complete | 1 Chromosome | 2888305 | 55.5 | 2458 | 2394 | 2382 | 64 | 3, | 52 | 3 |
| AMDK30 | | | | | | | | | 3, 3 | | |
| KGMB02009 | Complete | 1 Chromosome | 2844059 | 55.2 | 2395 | 2331 | 2314 | 64 | 3, | 52 | 3 |
| | | | | | | | | | 3, 3 | | |
| KGMB01988 | Complete | 1 Chromosome | 2844056 | 55.2 | 2393 | 2329 | 2312 | 64 | 3, | 52 | 3 |
| | | | | | | | | | 3, 3 | | |
| KGMB01989 | Complete | 1 Chromosome | 2844036 | 55.2 | 2392 | 2328 | 2311 | 64 | 3, | 52 | 3 |
| | | | | | | | | | 3, 3 | | |
| KGMB01990 | Complete | 1 Chromosome | 2844062 | 55.2 | 2391 | 2327 | 2310 | 64 | 3, | 52 | 3 |
| | | | | | | | | | 3, 3 | | |
| EB- | Complete | 1 Chromosome | 2799431 | 55.3 | 2377 | 2313 | 2279 | 64 | 3, | 52 | 3 |
| AMDK7 | | | | | | | | | 3, 3 | | |
| Akk1370 | Complete | 1 Chromosome | 2803677 | 55.3 | 2350 | 2286 | 2268 | 64 | 3, | 52 | 3 |
| | | | | | | | | | 3, 3 | | |
| Akk13715 | Complete | 1 Chromosome | 2803664 | 55.3 | 2348 | 2284 | 2259 | 64 | 3, | 52 | 3 |
| | | | | | | | | | 3, 3 | | |
| Akk1376 | Complete | 1 Chromosome | 2803683 | 55.3 | 2342 | 2278 | 2257 | 64 | 3, | 52 | 3 |
| | | | | | | | | | 3, 3 | | |
| Akk14745a | Complete | 1 Chromosome | 2798422 | 55.4 | 2337 | 2273 | 2253 | 64 | 3, | 52 | 3 |
| | | | | | | | | | 3, 3 | | |
| Akk14745b | Complete | 1 Chromosome | 2798160 | 55.4 | 2331 | 2267 | 2244 | 64 | 3, | 52 | 3 |
| | | | | | | | | | 3, 3 | | |
| Akk2670 | Complete | 1 Chromosome | 2761419 | 55.7 | 2323 | 2259 | 2244 | 64 | 3, | 52 | 3 |
| | | | | | | | | | 3, 3 | | |
| EB- | Complete | 1 Chromosome | 2772237 | 55.4 | 2326 | 2262 | 2231 | 64 | 3, | 52 | 3 |
| AMDK1 | | | | | | | | | 3, 3 | | |
| EB- | Complete | 1 Chromosome | 2770073 | 55.3 | 2346 | 2282 | 2231 | 64 | 3, | 52 | 3 |
| AMDK16 | | | | | | | | | 3, 3 | | |
| MGYG-HGUT- | Complete | 1 Chromosome | 2762447 | 55.2 | 2300 | 2236 | 2221 | 64 | 3, | 52 | 3 |
| 02454 | | | | | | | | | 3, 3 | | |
| Akk0096 | Complete | 1 Chromosome | 2755047 | 55.7 | 2294 | 2230 | 2218 | 64 | 3, | 52 | 3 |
| | | | | | | | | | 3, 3 | | |
| EB- | Complete | 1 Chromosome | 2736695 | 55.4 | 2282 | 2218 | 2202 | 64 | 3, | 52 | 3 |
| AMDK5 | | | | | | | | | 3, 3 | | |
| Akk0500a | Complete | 1 Chromosome | 2724325 | 55.6 | 2269 | 2205 | 2193 | 64 | 3, | 52 | 3 |
| | | | | | | | | | 3, 3 | | |
| Akk1713 | Complete | 1 Chromosome | 2724304 | 55.6 | 2273 | 2209 | 2193 | 64 | 3, | 52 | 3 |
| | | | | | | | | | 3, 3 | | |
| EB- | Complete | 1 Chromosome | 2724313 | 55.3 | 2277 | 2213 | 2188 | 64 | 3, | 52 | 3 |
| AMDK35 | | | | | | | | | 3, 3 | | |
| EB- | Complete | 1 Chromosome | 2724254 | 55.3 | 2277 | 2213 | 2187 | 64 | 3, | 52 | 3 |
| AMDK33 | | | | | | | | | 3, 3 | | |
| EB- | Complete | 1 Chromosome | 2724300 | 55.3 | 2277 | 2213 | 2184 | 64 | 3, | 52 | 3 |
| AMDK36 | | | | | | | | | 3, 3 | | |
| EB- | Complete | 1 Chromosome | 2724154 | 55.3 | 2306 | 2242 | 2182 | 64 | 3, | 52 | 3 |
| AMDK21 | | | | | | | | | 3, 3 | | |
| EB- | Complete | 1 Chromosome | 2724186 | 55.3 | 2288 | 2224 | 2177 | 64 | 3, | 52 | 3 |
| AMDK20 | | | | | | | | | 3, 3 | | |
| EB- | Complete | 1 Chromosome | 2724277 | 55.3 | 2259 | 2195 | 2176 | 64 | 3, | 52 | 3 |
| AMDK34 | | | | | | | | | 3, 3 | | |
| EB- | Complete | 1 Chromosome | 2724161 | 55.3 | 2299 | 2235 | 2168 | 64 | 3, | 52 | 3 |
| AMDK22 | | | | | | | | | 3, 3 | | |
| EB- | Complete | 1 Chromosome | 2724248 | 55.3 | 2259 | 2195 | 2159 | 64 | 3, | 52 | 3 |
| AMDK19 | | | | | | | | | 3, 3 | | |
| Akk0200 | Complete | 1 Chromosome | 2663997 | 55.8 | 2230 | 2166 | 2142 | 64 | 3, | 52 | 3 |
| | | | | | | | | | 3, 3 | | |
| AMUC | Complete | 1 Chromosome | 2664064 | 55.8 | 2202 | 2138 | 2132 | 64 | 3, | 52 | 3 |
| | | | | | | | | | 3, 3 | | |
| EB- | Complete | 1 Chromosome | 2664010 | 55.8 | 2218 | 2154 | 2130 | 64 | 3, | 52 | 3 |
| AMDK4 | | | | | | | | | 3, 3 | | |
| DSM | Complete | 1 Chromosome | 2664043 | 55.8 | 2203 | 2139 | 2124 | 64 | 3, | 52 | 3 |
| 22959 | | | | | | | | | 3, 3 | | |
| EB- | Complete | 1 Chromosome | 2663833 | 55.8 | 2263 | 2199 | 2123 | 64 | 3, | 52 | 3 |
| AMDK3 | | | | | | | | | 3, 3 | | |
| ATCC | Complete | 1 Chromosome | 2664102 | 55.8 | 2202 | 2138 | 2122 | 64 | 3, | 52 | 3 |
| BAA-835 | | | | | | | | | 3, 3 | | |

### 1.2. Differences in Genetic Characteristics Between Akkermansia Phylogroups

### 1.2.1. Phylotyping of 92 Human-Associated Akkermansia Genomes Through Phylogenetic Classification

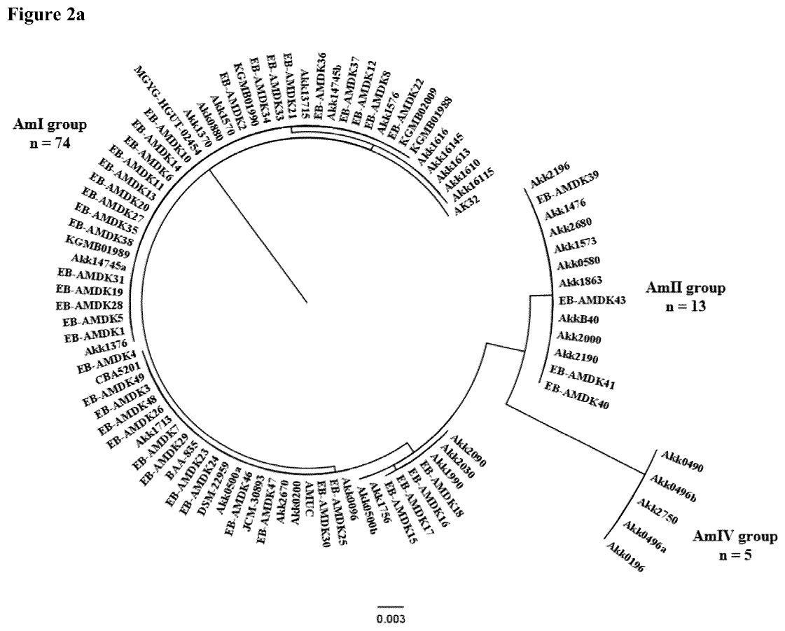

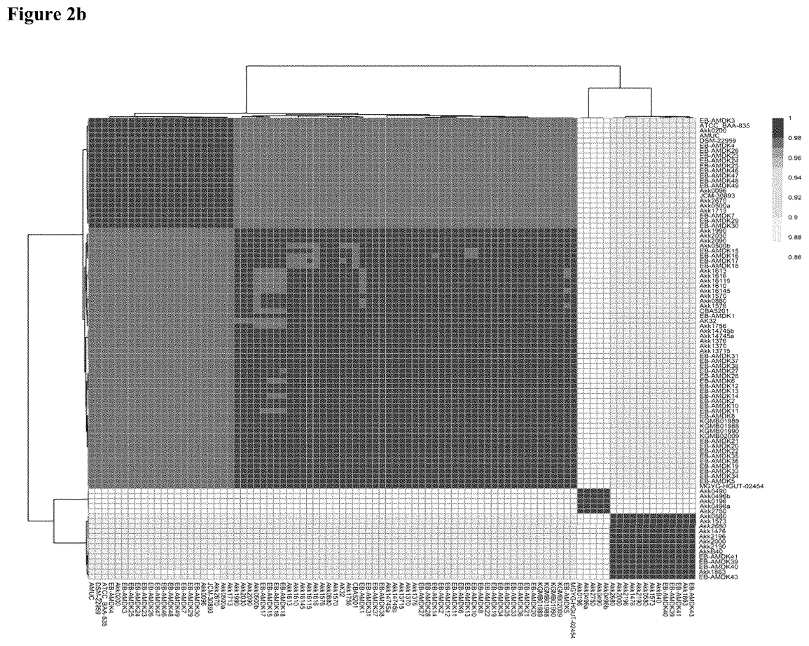

The phylogenetic classification of 92 human-associated Akkermansia genomes was performed by two methods using the 16S rRNA gene and the whole genome. The 16S rRNA genes were obtained from the obtained 92 human-associated Akkermansia genomes. The 16S rRNA genes obtained from 92 human-associated Akkermansia genomes were aligned using the clustal omega (v1.2.4) program. For the aligned sequences, phylogenetic classification was performed by applying the Neighbor-joining method in the MEGA11 program. As detailed options, Bootstrap method (1000) and Kimura 2-parameter model were applied and the derived phylogram is shown in FIG. 2a. In the case of phylogenetic classification based on the 92 human-associated Akkermansia whole genomes, the evolutionary distance was evaluated by applying the pyani v0.2.7 program with the -m ANIb setting and the results are shown in FIG. 2b.

FIGS. 2a and 2b show the results of phylogenetic classification of 92 human-associated complete Akkermansia genomes. FIG. 2a shows a phylogenetic classification based on the 16S rRNA sequence, and FIG. 2b shows a phylogenetic classification based on the whole genome. On the 16S rRNA sequence, it may be seen that 92 human-associated Akkermansia species are classified into three main phylogroups (AmI, AmII, and AmIV). It may be seen that 92 human-associated Akkermansia species are classified into three main phylogroups (AmI, AmII, and AmIV) even in the phylogenetic classification based on the whole genome. Among the 92 human-associated Akkermansia species, 74 were classified as AmI, 13 as AmII, and 5 as AmIV. It was confirmed that the AmI phylogroup including the largest number of Akkermansia in the phylogenetic classification based on the whole genome was subdivided into AmIa (22, including BAA-835 strain) and AmIb (52, including EB-AMDK19 strain) based on the average nucleotide identity (ANI) value of 97%.

Since the 92 human-associated Akkermansia genomes are classified into three main phylogroups in the phylogenetic classification based on the 16S rRNA gene sequence, it was confirmed whether there is an identification region on the 16S rRNA gene to specifically identify the phylogroup. Specifically, the 16S rRNA genes of the 92 human-associated Akkermansia species were aligned using the clustal omega (v1.2.4) program, and classification was attempted for each phylogroup, thereby confirming phylogroup-specific identification regions which are constant in the phylogroup and different between the phylogroups (see FIG. 1 and Table 2). In addition, in order to confirm the specificity of the phylogroup-specific identification region, intra-phylogroup or inter-phylogroup genetic difference for each phylogroup-specific identification region was evaluated. The evaluation of genetic difference was calculated through the similarity (%) derived from the blastn-short setting of the blastn program.

As a result of intra-phylogroup or inter-phylogroup genetic evaluation for phylogroup identification region 1, a genetic difference of 0.270±0.731% was confirmed in the AmI phylogroup. No genetic difference was confirmed in the AmII phylogroup or in the AmIV phylogroup. In the inter-phylogroup comparison, the AmI phylogroup and the AmII phylogroup showed a genetic difference of 5.814±0.000%, the AmI phylogroup and the AmIV phylogroup showed 8.642±0.000%, and the AmII phylogroup and the AmIV phylogroup showed 11.111±0.000%. As a result of intra-phylogroup or inter-phylogroup genetic evaluation for phylogroup identification region 2, no genetic difference was confirmed in the phylogroup, which is the same. In the inter-phylogroup comparison, the AmI phylogroup and the AmII phylogroup showed a genetic difference of 4.545±0.000%, the AmI phylogroup and the AmIV phylogroup showed 5.128±0.000%, and the AmII phylogroup and the AmIV phylogroup showed 2.564±0.000%. In the case of phylogroup identification region 3, a genetic difference through the blastn program could not be confirmed because it consists of 12 short base sequences. However, it was confirmed that phylogroup identification region 3 shows a sequence constant in the phylogroup and specifically different sequences between the phylogroups.

### 1.2.2. Genetic Distance Based on Comparison in the Same Phylogroup or Comparison Between Different Phylogroups

It was intended to confirm the intra-phylogroup or inter-phylogroup genetic difference of Akkermansia based on the whole genome or the core genome. In order to derive the intra-phylogroup or inter-phylogroup genetic distance based on the whole genome, the average nucleotide identity (ANI) value was derived by applying the pyani v0.2.7 program with the -m ANIb setting in the phylogroup or between the phylogroups. The whole genome genetic distance in the phylogroup or between the phylogroups was calculated by inversely taking the derived similarity (%) value and the results are shown in Table 4. In deriving the genetic distance in the phylogroup or between the phylogroups based on the core genome, Roary (v3.11.2), a high-speed stand alone pan genome pipeline, was used. Specifically, in order to evaluate the genetic distance by core gene alignment, Roary analysis was performed by taking annotated assemblies from GFF3 format produced by Prokka (v1.13.4). The core genome alignment sequence derived through Roary analysis was classified for each phylogroup, and the similarity (%) in the phylogroup or between the phylogroups was calculated through the blastn program. The core genome genetic distance in the phylogroup or between the phylogroups was calculated by inversely taking the derived similarity (%) value and the results are shown in Table 4.

As shown in Table 4 below, it may be confirmed that the genetic distance in the same phylogroup is very low, less than 2%, whereas the genetic distance between other phylogroups corresponds to 12-18%. From above, it may be confirmed that Akkermansia exhibits a specific genetic distance according to the classified phylogroup. In other words, it means that Akkermansia is clearly divided into three main phylogroups.

TABLE 4

| Intra-phylogroup/ | Whole genome | Core genome |
|---|---|---|
| Inter-phylogroup | distance (%) | distance (%) |
| AmI | 1.892 ± 0.747 | 1.291 ± 0.532 |
| AmII | 0.878 ± 0.620 | 0.652 ± 0.473 |
| AmIV | 0.057 ± 0.026 | 0.017 ± 0.010 |
| AmI vs AmII | 12.224 ± 0.121 | 9.469 ± 0.049 |
| AmI vs AmIV | 18.105 ± 0.063 | 14.663 ± 0.052 |
| AmII vs AmIV | 16.565 ± 0.090 | 12.507 ± 0.037 |

### 1.3. Preparation of Akkermansia Phylogroup-Specific Primer

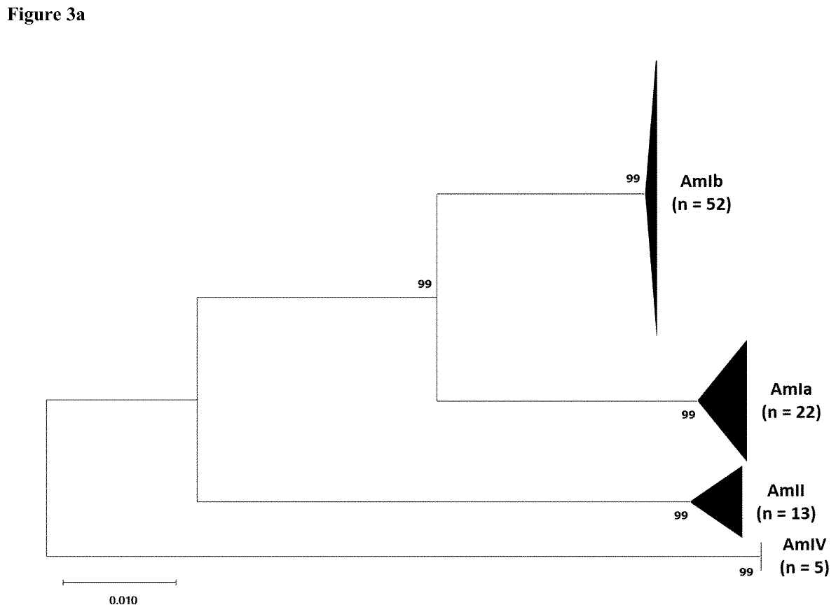

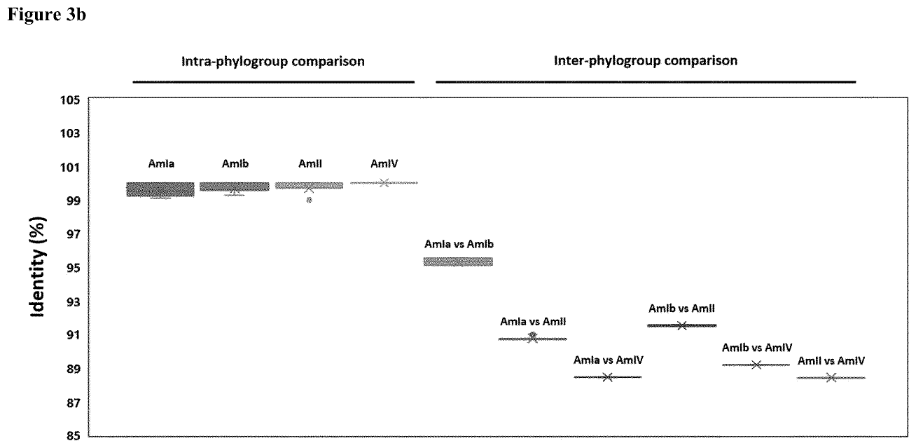

Marker genes, which exist in a single copy on all the Akkermansia whole genomes and show distinct differences between the phylogroups, were searched. Based on the 92 human-associated Akkermansia whole genomes obtained in Examples above, an orthologous gene search was performed using get homologues software (https://github.com/eead-csic-compbio/get homologues). Specifically, for the genbank file format with respect to the 92 Akkermansia whole genomes, orthologous genes were searched based on the OrthoMCL algorithm as clustering criteria. Among the searched genes, the gene, which covers all of the Akkermansia phylogroups (AmIa, AmIb, AmII, and AmIV) and exists in a single copy, was obtained. The obtained gene is sodium ion-translocating decarboxylase subunit beta, and was aligned using the clustal omega (v1.2.4) program after obtaining the corresponding gene sequence from the 92 Akkermansia whole genomes. For the aligned sequences, phylogenetic classification was performed by applying the Neighbor-joining method in the MEGA11 program. As detailed options, Bootstrap method (1000) and Kimura 2-parameter model were applied and the derived phylogram is shown by FIG. 3(A). Additionally, in order to confirm whether the obtained gene exhibits a specific genetic difference between the phylogroups, the similarity (%) in the phylogroup or between the phylogroups was calculated through the blastn program and the results are shown in FIG. 3(B).

As shown in FIG. 3(A), it may be confirmed that the 92 Akkermansia whole genomes are classified according to the phylogroups (AmIa, AmIb, AmII, and AmIV) in the phylogenetic classification based on the sodium ion-translocating decarboxylase subunit beta gene obtained through the orthologous gene analysis. This result indicates that the sodium ion-translocating decarboxylase subunit beta gene can be utilized as a marker gene. As shown in FIG. 3(B), the sodium ion-translocating decarboxylase subunit beta gene, which is a marker gene, shows high similarity in comparison of similarity in the Akkermansia phylogroup, whereas it shows low similarity in comparison of similarity between the Akkermansia phylogroups. As a result, it was confirmed that the sodium ion-translocating decarboxylase subunit beta gene, which is a marker gene, is a gene suitable for designing a phylogroup-specific primer.

In order to design a phylogroup-specific primer based on the sodium ion-translocating decarboxylase subunit beta which is the marker gene, the marker gene sequence was obtained from the 92 Akkermansia whole genomes, and then was aligned using the clustal omega (v1.2.4). The conservative gene region and highly variable gene region were identified for each Akkermansia phylogroup. Phylogroup-specific primers as shown in Table 5 below were prepared through the blastn program in the highly variable gene region.

### Example 2: Confirmation of Distribution Pattern of Human Gut Akkermansia Phylogroups

### Example 2.1: Phylotyping of Human Gut Akkermansia Based on Gut Microbiota Analysis

As identification regions which allow for specifically identifying the Akkermansia phylogroup on the 16S rRNA gene were established (see Table 2 and FIG. 1), it was intended to perform phylotyping of human gut Akkermansia from publicly available metagenome data. The metagenome data of 890 Koreans utilized in the present invention were obtained from the NCBI database (BioProject: PRJEB33905). The sequencing data for the 16S rRNA gene was converted to an ASV frequency table. The ASV table was created through the DADA2 pipeline of the QIIE2 program (version 2019.01). On the obtained ASV table, the ASV corresponding to Akkermansia was extracted and aligned with the 16S rRNA gene sequence obtained from the whole genomes of the 92 human-associated Akkermansia strains. Based on this, phylotyping for each ASV was performed from the Akkermansia phylogroup identification region.

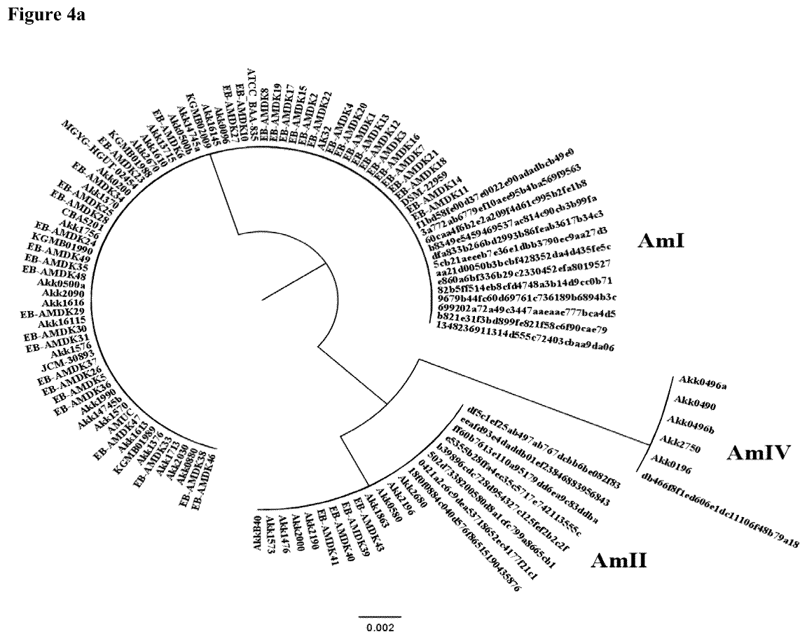

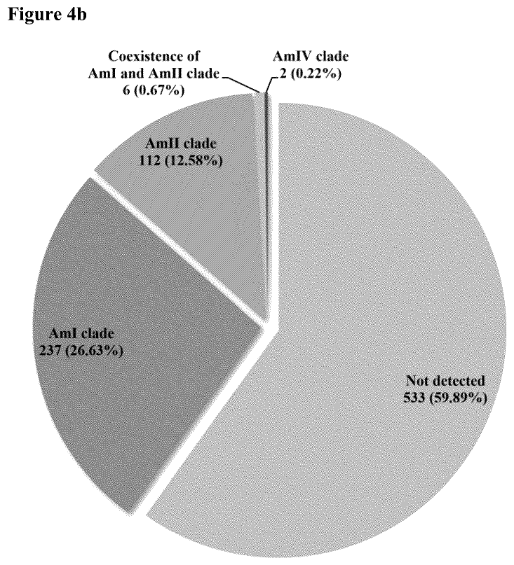

The present inventors confirmed phylogroups of Akkermansia on the 16S rRNA V3-V4 regions, or that the phylogroups can be divided (see FIG. 4a). It was confirmed that 13 of the 22 ASVs corresponding to Akkermansia were AmI, 8 were AmII, and 1 was AmIV (see FIG. 4a). Based on this, the ratio according to the presence or absence of the gut Akkermansia of 890 Korean and the ratio according to the phylogroups when the gut Akkermansia existed were analyzed, and the results are shown in FIG. 4b. Referring to FIG. 4b, it was confirmed that Akkermansia does not exist in a complex form consisting of a plurality of phylogroups, but exists in a form in which a single phylogroup predominates. In addition, it was confirmed that the case of the co-existence of AmI and AmII phylogroups was extremely rare, less than 1%. It was confirmed that AmIV also exists very rarely.

### 2.2. Confirmation of Distribution Pattern of Human Gut Akkermansia Phylogroups in Various Countries

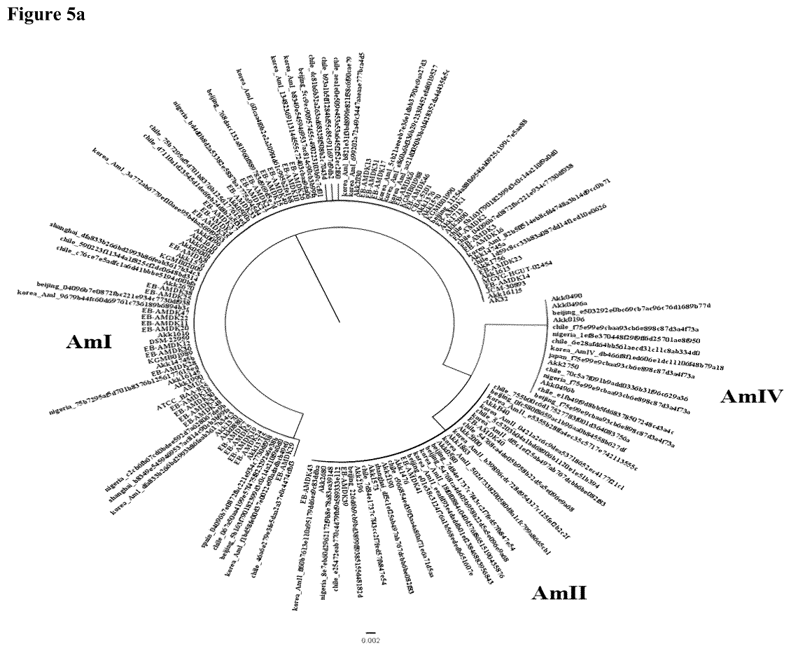

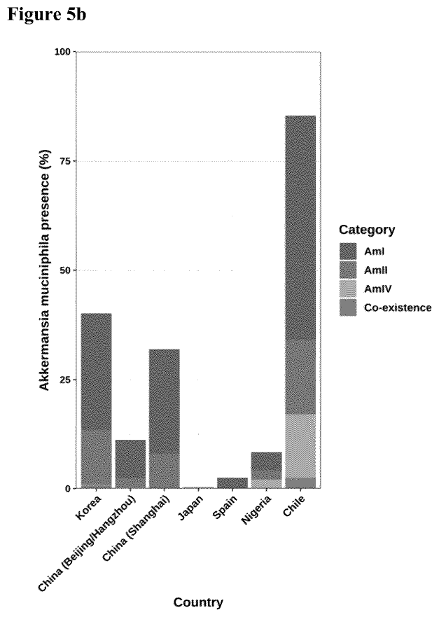

Metagenome data from various countries other than Korea were obtained from the NCBI database and the MG-RAST database. Specifically, Chilean metagenome data were obtained from PRJEB16755, Nigerian metagenome data from mgp83994, Chinese (Beijing) metagenome data from PRJNA480547, Chinese (Shanghai) metagenome data from PRJNA382861, Japanese metagenome data from PRJDB4360, and Spanish metagenome data from PRJNA350839. The analysis of the metagenome data and identification of the Akkermansia phylogroups were performed in the same manner as described in Example 2.1, and the results are shown in FIG. 5.

Referring to FIG. 5, the case of the coexistence of AmI and AmII was rarely observed. In other words, it was confirmed that the unique distribution pattern of Akkermansia, which is the dominant pattern of a single phylogroup based on the feature point that the coexistence of AmI and AmII is shown extremely low, is the characteristic of Akkermansia, which is derived not only from Koreans but also from various countries. From these results, it was confirmed that the exclusion phenomenon between the Akkermansia phylogroups AmI and AmII was shown not only in Korea but also in various countries. That is, it was confirmed that the approach of the present invention may be used not only in Korea but also in various countries.

### 3. Confirmation of Specific Exclusion Pattern Between Akkermansia Phylogroups

### 3.1. Identification of Competitive Exclusion Relationships Between Akkermansia Phylogroups

Cell-free supernatants derived from strains representing each phylogroup of Akkermansia were used to analyze their influence on each other's growth. For this purpose, Akkermansia EB-AMDK19 strains (AmI phylogroup), Akkermansia EB-AMDK39 strains (AmII phylogroup), and Akkermansia EB-ABDH76 strains (AmIV phylogroup) were inoculated in the culture medium (30 g/L of tryptic soy broth (TSB), 2.5 g/L of mucin from porcine stomach, 0.1 mg/L of cyanocobalamin, and 0.5 g/L of L-cysteine hydrochloride) and cultured for 24 hours. The supernatant and strain pellet were separated through the centrifugation process (10,000 rpm, 10 minutes, 4° C.). A cell-free supernatant was prepared by filtering the separated supernatant through a 0.2 μm syringe filter. To determine the effect of the cell-free supernatant obtained from each Akkermansia phylogroup on other types of Akkermansia phylogroup, when 0.1% of the strain corresponding to each phylogroup was inoculated into the culture medium, 20% (v/v) of the culture medium was inoculated. The effect on growth of other phylogroup strains was determined by comparing the absorbance value after 24 hours culture with the absorbance value of the control group (see Table 5).

TABLE 5

| | Growth (% of medium control) | | |
|---|---|---|---|
| Additive | AmI | AmII | AmIV |
| (20%, v/v) | (EB-AMDK19) | (EB-AMDK39) | (EB-ABDH76) |
| AmI (EB-AMDK19) | 92.39 ± 6.09 | 101.56 ± 6.02 | 100.16 ± 2.36 |
| derived supernatant | | | |
| AmII (EB-AMDK39) | 9.64 ± 4.06 | 102.36 ± 3.40 | 97.20 ± 5.72 |
| derived supernatant | | | |
| AmIV (EB-ABDH76) | 1.33 ± 0.14 | 1.25 ± 0.36 | 97.22 ± 0.63 |
| derived supernatant | | | |

Referring to the above Table 5, the cell-free supernatant derived from Akkermansia EB-AMDK19 strain, a representative strain of the Akkermansia phylogroup AmI had no effect on the growth of Akkermansia EB-AMDK39 strain, a representative strain of the Akkermansia phylogroup AmII, and Akkermansia EB-ABDH76 strain, a representative strain of the Akkermansia phylogroup AmIV. Cell-free supernatant derived from Akkermansia EB-AMDK39 strain, a representative strain of the Akkermansia phylogroup AmII, specifically inhibited the growth of Akkermansia EB-AMDK19 strain, but did not affect the growth of Akkermansia EB-ABDH76 strain. Cell-free supernatant derived from Akkermansia EB-ABDH76 strain, a representative strain of the Akkermansia phylogroup AmIV, specifically inhibited the growth of Akkermansia EB-AMDK19 strain and Akkermansia EB-AMDK39 strain.

### 3.2. Confirmation of Specific Inhibition Patterns Between Phylogroups

In order to verify whether representative strains of each phylogroup can show specific inhibition patterns between phylogroup groups, 14 strains of the AmI phylogroup and 3 strains of the AmII phylogroup were additionally selected and inhibition patterns between phylogroups were analyzed by phylogenetic classification based on the full-length genome (FIG. 2). Specifically, four strains of the AmII phylogroup (Akkermansia EB-AMDK39, EB-AMDK40, EB-AMDK41, EB-AMDK43) were inoculated in the culture medium (30 g/L of tryptic soy broth (TSB), 2.5 g/L of porcine stomach mucin, 0.1 mg/L of cyanocobalamin, and 0.5 g/L of L-cysteine hydrochloride) at 0.1% and cultured for 24 hours. The supernatant and the strain pellet were separated by centrifugation (10,000 rpm, 10 minutes, 4° C.). A cell-free supernatant was prepared by filtering the separated supernatant through a 0.2 m syringe filter. To determine the effect of the cell-free supernatant obtained from phylogroup AmII strains on the growth of 15 AmII AmI strains and 4 AmII phylogroup strain, when 0.1% of the strain corresponding to each phylogroup was inoculated into the culture medium, 20% (v/v) of the culture medium was inoculated. The effect on growth of other phylogroup strains was determined by comparing the absorbance value after 24 hours culture with the absorbance value of the control group (see Table 6).

TABLE 6

| | | Additive (20%, v/v) | | | |
|---|---|---|---|---|---|
| | | EB-AMDK39 | EB-AMDK40 | EB-AMDK41 | EB-AMDK43 |
| | | derived | derived | derived | derived |
| phylogroup | Strain | supernatant | supernatant | supernatant | supernatant |
| AmI | BAA-835^{T} | 4.37 ± 0.47 | 3.76 ± 0.18 | 3.86 ± 0.63 | 2.34 ± 0.18 |
| | EB-AMDK3 | 7.69 ± 1.95 | 2.75 ± 0.98 | 7.61 ± 1.84 | 4.94 ± 0.56 |
| | EB-AMDK4 | 6.57 ± 1.20 | 10.43 ± 2.49 | 9.90 ± 0.55 | 9.20 ± 1.72 |
| | EB-AMDK5 | 8.26 ± 1.30 | 7.59 ± 3.31 | 6.75 ± 1.83 | 4.97 ± 0.53 |
| | EB-AMDK6 | 8.05 ± 0.16 | 5.71 ± 0.58 | 5.24 ± 0.65 | 3.65 ± 0.28 |
| | EB-AMDK7 | 5.86 ± 0.34 | 3.67 ± 0.34 | 3.57 ± 0.52 | 7.94 ± 1.89 |
| | EB-AMDK8 | 4.98 ± 0.90 | 3.95 ± 0.15 | 4.03 ± 0.54 | 3.86 ± 1.29 |
| | EB-AMDK10 | 8.94 ± 0.79 | 5.61 ± 0.66 | 4.91 ± 0.30 | 6.57 ± 0.79 |
| | EB-AMDK19 | 5.46 ± 3.25 | 5.54 ± 0.52 | 3.72 ± 0.43 | 4.63 ± 0.94 |
| | EB-AMDK23 | 3.19 ± 0.05 | 3.29 ± 0.17 | 3.24 ± 0.12 | 3.59 ± 0.08 |
| | EB-AMDK27 | 3.68 ± 0.36 | 4.10 ± 0.32 | 4.21 ± 0.66 | 3.68 ± 0.18 |
| | EB-AMDK29 | 5.30 ± 0.84 | 3.74 ± 0.15 | 4.08 ± 0.54 | 7.99 ± 1.44 |
| | EB-AMDK31 | 3.46 ± 0.04 | 4.41 ± 1.64 | 3.15 ± 0.18 | 7.45 ± 0.18 |
| | EB-AMDK37 | 2.77 ± 0.33 | 3.24 ± 0.17 | 2.96 ± 0.44 | 3.15 ± 0.04 |
| | EB-AMDK46 | 4.06 ± 0.54 | 4.23 ± 0.30 | 3.62 ± 0.52 | 5.44 ± 0.45 |
| AmII | EB-AMDK39 | 99.92 ± 3.57 | 93.63 ± 4.87 | 96.07 ± 4.58 | 98.11 ± 4.95 |
| | EB-AMDK40 | 105.19 ± 3.11 | 97.56 ± 2.67 | 94.05 ± 1.60 | 94.74 ± 4.14 |
| | EB-AMDK41 | 98.82 ± 4.03 | 100.08 ± 6.13 | 102.29 ± 1.88 | 95.66 ± 2.69 |
| | EB-AMDK43 | 95.25 ± 0.45 | 97.81 ± 3.49 | 93.52 ± 2.51 | 96.67 ± 1.20 |

Referring to the above Table 6, all cell-free supernatants derived from the four phylogroup AmII strains specifically inhibited the growth of the 15 phylogroup AmI strains (2-10% of medium control). Additionally, none of the cell-free supernatants derived from the four phylogroup AmII strains had any specific effect on the growth of the four phylogroup AmII strains (>93% of medium control). Through this, it was confirmed that representative strains (Akkermansia EB-AMDK19, EB-AMDK39, EB-ABDH76) for each phylogroup showed specific inhibition patterns between the Akkermansia phylogroup.

### 3.3. Confirmation of Settlement in Guts of Mice According to Single Administration for Each Akkermansia Phylogroup

All animal experiments were carried out in accordance with the guidelines of the Institutional Animal Care and Use Committee (IACUC). Specifically, germ-free mice (C57BL/6) were raised and kept in sterile flexible film isolators (Class Biological Clean Ltd.) in conditions of 23° C., relative humidity (40-60%), and a 12-hour light/dark cycle. The six-week-old female germ-free mice (C57BL/6) raised and kept in the above conditions were randomly divided into three groups (n=4) as shown in Table 7 below. The germ-free mice applied in the experiment were routinely monitored for microbial contamination by culturing fresh fecal samples under aerobic and anaerobic conditions.

In order to confirm whether each Akkermansia phylogroup in the germ-free mice is colonized, Akkermansia muciniphila BAA-835 was selected as a strain representing Akkermansia phylogroup AmIa, Akkermansia muciniphila EB-AMDK19 was selected as a strain representing AmIb and Akkermansia muciniphila EB-AMDK39 was selected as a strain representing AmII and applied to the experiment. Frozen stock vials were prepared at a concentration of 1×10⁸ CFU of live bacteria per 150 μL of PBS containing 25% glycerol and 0.05% cysteine for the representative strain of each of the aforementioned phylogroups.

Using the frozen stock vials prepared by the above method, 150 μL of the live bacteria (1×10⁸ CFU) of the representative strain of each phylogroup (Akkermansia muciniphila BAA-835, Akkermansia muciniphila EB-AMDK19, and Akkermansia muciniphila EB-AMDK39) was orally administered to each experimental group over a total of two days once a day (see Table 7). After the oral administration, fresh feces for each experimental group were periodically collected and stored in a −80° C. freezer for use in determining whether each Akkermansia phylogroup was colonized.

TABLE 7

| Experimental groups | Administration information |
|---|---|
| Experimental | Administration group of BAA-835 live bacteria |
| Group I | (1 × 10⁸ CFU) representing AmIa phylogroup |
| Experimental | Administration group of EB-AMDK19 live bacteria |
| Group II | (1 × 10⁸ CFU) representing AmIb phylogroup |
| Experimental | Administration group of EB-AMDK39 live bacteria |
| Group III | (1 × 10⁸ CFU) representing AmII phylogroup |

In order to confirm the analytical sensitivity and specificity of the Akkermansia phylogroup-specific primer shown in Table 1, a DNA fragment including the DNA sequence of each target marker was prepared.

TABLE 8

| | Sequence of DNA fragment containing DNA sequence | |
|---|---|---|
| Target | of each target marker (5′→3′) | SEQ ID NO. |
| AmIa | CGCTTCAGCAGGCTCCCTCCAAGGTGCCGGCCAATCTGACCA | 18 |
| | CGCCTGAATCCCGTGCCCAGTATCAGGAAATCATGCAGCAGC | |
| | CCATGCAGGTTTACCCCGGCAGCCAGCTGACCGTTTCCAAGA | |
| | TCAAGTCCGTGCGGGAATCCCAGGAAAAAGCAAAAGCTGAC | |
| | GCGGCCCGCCTGGGCGACGACAGCCTGACGGTGGACCCCAA | |
| | CCTGAAGGATTTCCAGAACGTGACGGAAGACAACGGCAATG | |
| | AACC | |
| AmIb | ATGTGGCTCCTCTGCAGCAGACGCCCGCCAAGGTGCCGGCCA | 19 |
| | ATCTGACCACGCCGGAAGCCCGCGCCCAGTATCTGGAAACCA | |
| | TGCAGCAGCCCATGCAGGTTTATCCCGGCAGCCAGCTGACCG | |
| | TTTCCAAAATCAAGTCCGTGCGGGAATCCCAGGAAAAAGCC | |
| | AAAGCTGACGCCGCCCGCCTGGGAGACGACAGTCTGACGGT | |
| | GGATCCCAACCTGAAGGACTTCCAGAACGTGACGGAGGACA | |
| | ACGGCAATGAGCCGGTCTTCCTGCTCACGAACGGAGAAGGA | |
| | ACCACCGTCGTCCGCCAGCAGGGCGTCAATTACTTTGACACC | |
| | AGCGGCAACCGTGTTCCCGTGGACCT | |
| AmII | TTGGCGCATTAATTGCCAACATTCCGGACAACGGAATGCTCA | 20 |
| | TCACCCAGCTGAACCAGCAGGTCATCTCCTCCAATAAGGCGG | |
| | GGGAAGTGACCGCCACGTCCCTCAACAACGTGGGCTACCTGC | |
| | GCGTTCACGTGGCCCCGCTCCAGCAGGCCCCCGTCAAGGTGC | |
| | CGGCCAACCTGACCACGCCTGAAGCGCGCGCCCAGTATCTGG | |
| | CAAACATGGAACAGCCCATGCAGGTTTACCCCGGCAGCCCGC | |
| | TGACCGTCTCCCAGATCAAGCCCGTGCGGGAATCCCAGGAAA | |
| | AAGCCAAGGCTGACGCCGCCCTTCTGGGGGACGACAGCCTG | |
| | ACGGTGGACCCCAACCTGAAGGACTTCCACAACGTGACGGA | |
| | AGCGGACGCCAACGATCCGGTGTACCTGCTCACGAACGGTG | |
| | GAGTCACCACCGTTCTGCGCCAGAAGGGAGTCAACTACTTTG | |
| | ACACGGA | |

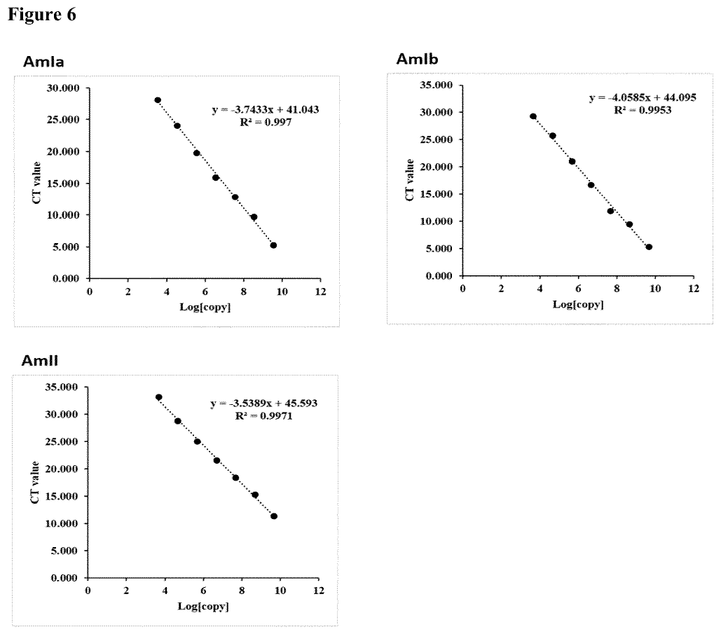

The prepared DNA fragment was subjected to serial dilution (10³-10⁹) and quantitative PCR was performed using the DNA fragment as a template to test the analytical sensitivity. Quantitative PCR experiments were performed using quantitative PCR kits (TOPreal SYBR Green High-ROX PreMIX, Enzynomics) and ABI Quantstudio 3 Real-Time PCR Instrument, 96-well, 0.2 mL (A28132). As a result, it was confirmed that the performance of the Akkermansia phylogroup-specific primer (Amla, AmIb, and AmII) quantifies each Akkermansia phylogroup in a concentration-dependent manner (see FIG. 6)

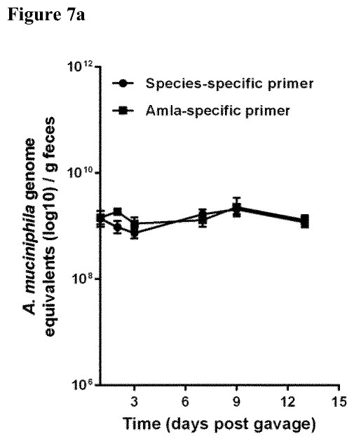

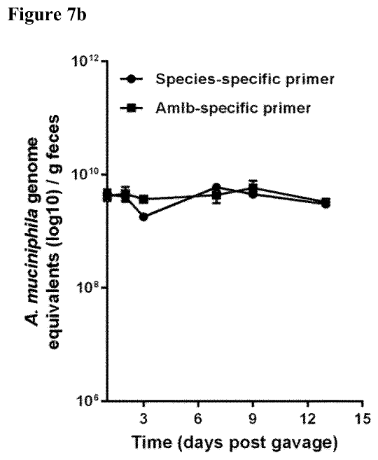

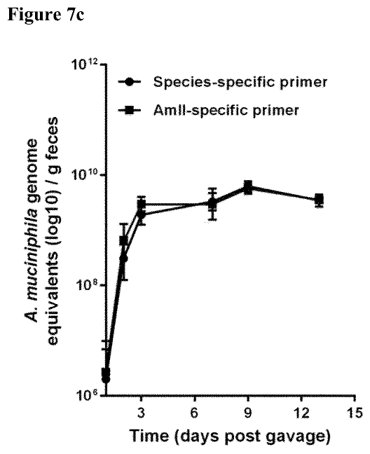

Then, the DNA was extracted by orally administering the live bacteria of the representative strain of each Akkermansia phylogroup and then applying a fecal DNA extraction kit (QIAamp PowerFecal Pro DNA Kit, QIAGEN) to fresh feces collected periodically for each experimental group. Quantitative PCR was performed using each of the Akkermansia phylogroup-specific primers in Table 1 and quantitative PCR kits (TOPreal SYBR Green High-ROX PreMIX, Enzynomics), thereby determining changes in the level of each Akkermansia phylogroup and retention patterns of settlement in feces after administration. Specifically, 9 μL of DNA template, L of PreMIX, and 10 pmol of phylogroup-specific primer per each well were used, and thus a total of 20 μL was reacted. In this case, the PCR conditions were as follows: a cycle of initial 50° C. for 4 minutes, 95° C. for 10 minutes, 95° C. for 30 seconds, and 56° C. for 30 seconds was repeated 40 times. The CT value for each experimental group at each fecal collection time point derived through quantitative PCR was substituted into the trend line for each Akkermansia phylogroup-specific primer of FIG. 6, so that the changes in the level of each Akkermansia phylogroup and retention patterns of settlement in feces were identified per gram feces, and the results are shown in FIGS. 7a and 7b.

As shown in FIGS. 7a-7c (FIG. 7a: BAA-835 administration, FIG. 7b: EB-AMDK19 administration, FIG. 7c: EB-AMDK39 administration), it may be confirmed that when strains for each Akkermansia phylogroup are administered alone, all strains for each phylogroup are observed at a high level after the administration. In addition, values of genome equivalents (log 10)/g feces specific to each phylogroup-specific primer, which are increased according to administration, are retained, and thus it may also be confirmed that the level of settlement persists.

### 3.4. Analysis of Settlement Patterns in Guts of Mice According to Coadministration of Akkermansia Phylogroups

Six-week-old female germ-free mice (C57BL/6) were randomly divided into two groups (n=4) as shown in Table 7 below. Frozen stock vials were prepared at a concentration of 1×10⁸ CFU of live bacteria per 150 μL of PBS containing 25% glycerol and 0.05% cysteine for the representative strain of each Akkermansia phylogroup to be used in the experiment.

Using the stock vials prepared by the above method, 150 μL of the live bacteria (1×10⁸ CFU) of the representative strain of each Akkermansia phylogroup (BAA-835, EB-AMDK19, and EB-AMDK39) was orally administered to experimental groups over a total of two days once a day (see Table 9). After the oral administration, fresh feces for each experimental group were periodically collected and stored in a −80° C. freezer in order to confirm changes in the gut level for each Akkermansia phylogroup.

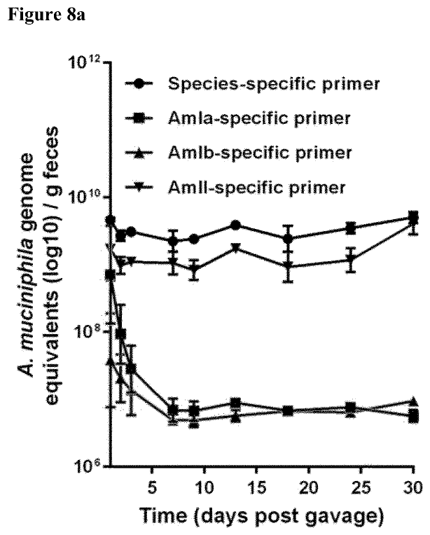

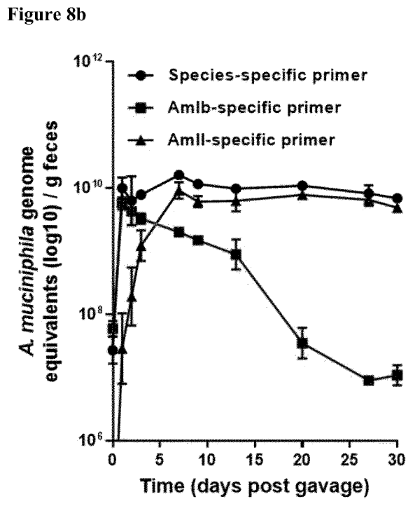

Changes in the gut level for each Akkermansia phylogroup according to the coadministration of the Akkermansia phylogroups were confirmed by performing quantitative PCR on gDNA extracted from fresh feces collected for each experimental group at each time point after the coadministration of the Akkermansia phylogroups, and the results are shown in FIGS. 8a and 8b.

TABLE 9

| Experimental | |
|---|---|
| groups | Administration information |
| Experimental | Co-administration group of BAA-835 live bacteria |
| Group I | (1 × 10⁸ CFU) representing AmIa phylogroup, |
| | EB-AMDK19 live bacteria (1 × 10⁸ CFU) representing |
| | AmIb phylogroup, and EB-AMDK39 live bacteria |
| | (1 × 10⁸ CFU) representing AmII phylogroup |
| Experimental | Co-administration group of EB-AMDK19 live bacteria |
| Group II | (1 × 10⁸ CFU) representing AmIb phylogroup and |
| | EB-AMDK39 live bacteria (1 × 10⁸ CFU) representing |
| | AmII phylogroup |

Referring to FIG. 8a, it may be seen that when various Akkermansia phylogroups (Akkermansia muciniphila BAA-835: AmIa, EB-AMDK19: AmIb, EB-AMDK39: AmII) were coadministered, the AmIa and AmIb phylogroups belonging to the AmI phylogroup continuously decreases after the administration and reaches the detection limit line. On the other hand, high-level values of genome equivalents (log 10)/g feces specific to the AmII phylogroup primer are retained from the administration until the end of the experiment, and thus it may be confirmed that the AmII phylogroup shows competitive superiority in the gut to the AmI phylogroup.

Referring to FIG. 8b, when two types of Akkermansia phylogroups AmIb and AmII are coadministered, as the results in FIG. 8(A), the AmIb belonging to the AmI phylogroup continuously decreases after the administration and reaches the detection limit line. On the other hand, in the AmII phylogroup, a high level of existence pattern was found in the feces from the administration to the end of the experiment. Accordingly, it may be seen that a competitive relationship for settlement in the host intestinal tract is formed between the AmI and AmII phylogroups.

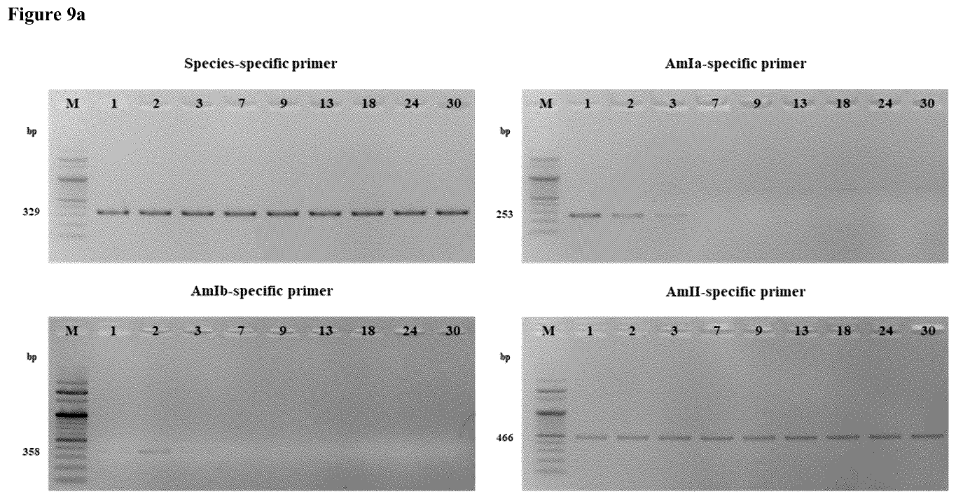

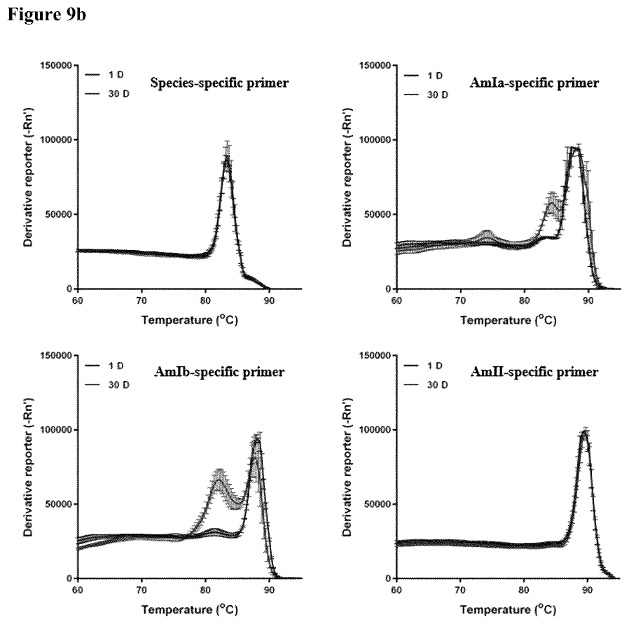

The specificity of the Akkermansia phylogroup-specific primer was confirmed from the quantitative PCR analysis. The PCR product was electrophoresed after the completion of quantitative PCR in which phylogroup-specific primers were applied to the gDNA extracted from the feces at each time point obtained according to the coadministration of various Akkermansia phylogroups. The electrophoresis was performed by loading the PCR product on a 2.0% agarose gel containing RedSafe Nucleic Acid Staining Solution (20,000×) made by iNtRON Biotechnology, Inc. The results were read by comparing the bands shown after the electrophoresis with amplicon sizes (bp) of the Akkermansia phylogroup-specific primers (FIG. 9a).

In addition, the specificity of the Akkermansia phylogroup-specific primer was confirmed from the melting curve plots derived through the quantitative PCR analysis. Changes in the melting curve plots were observed after the completion of quantitative PCR in which phylogroup-specific primers were applied to the gDNA extracted from the feces at each time point obtained according to the coadministration of various Akkermansia phylogroups. For the melting curve plot, the results were read by comparing the melting curve plot at the end of the experiment with the melting curve plot on day 1 of the administration (FIG. 9b).

FIG. 9a shows the electrophoresis results of quantitative PCR products using Akkermansia species-specific and phylogroup-specific primers. It was confirmed that the single amplification band pattern by the phylogroup-specific primers is retained even on the electrophoresis as the AmII phylogroup exhibits a high level from the beginning of administration to the end of the experiment as seen in the results of FIG. 9a. On the other hand, it may be confirmed that the AmIa and AmIb phylogroups belonging to the AmI phylogroup rapidly decrease immediately after the administration, and thus the single amplification band pattern disappears on the electrophoresis. In addition, the above matters may be equally confirmed on the melting curve plots when performing quantitative PCR using the Akkermansia species-specific and phylogroup-specific primers of FIG. 9b. Since the AmII phylogroup retains a dominant position from the time immediately after the administration to the end of the experiment, it may be seen that the melting curve plots at the beginning and end of the experiment are suitable and consistent. On the other hand, it may be seen that as the AmIa and AmIb phylogroups rapidly decrease, the melting curve plot at the end of the experiment is not consistent with that at the beginning of the experiment, and shows a pseudo-positive melting curve plot. Through the above matters, the specificity of the Akkermansia phylogroup-specific primer may be confirmed.

### 3.5. Analysis of Settlement Patterns in Guts of Mice According to Cross-Administration of Akkermansia Phylogroups

When another type of Akkermansia phylogroup is orally administered in the state in which the gut Akkermansia phylogroup is specified, in order to confirm change patterns in each gut Akkermansia phylogroup, 6-week-old female germ-free mice (C57BL/6) were randomly divided into two groups (n=4) as shown in Table 8 below. Frozen stock vials were prepared at a concentration of 1×10⁸ CFU of live bacteria per 150 μL of PBS containing 25% glycerol and 0.05% cysteine for the representative strain of each Akkermansia phylogroup to be used in the experiment.

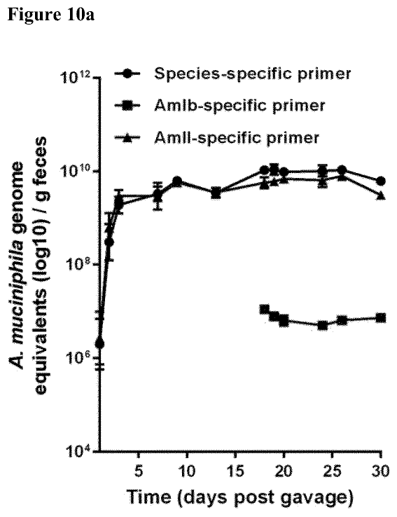

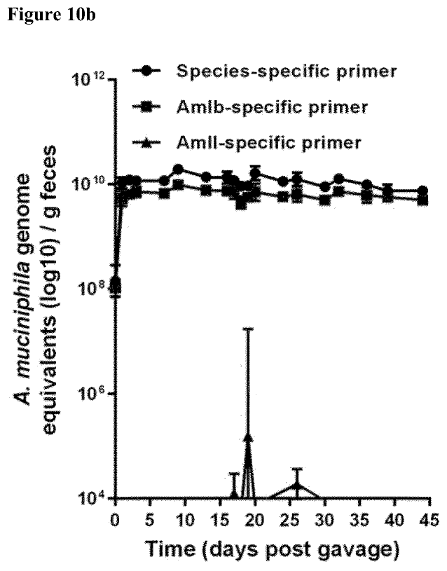

Using the stock vials prepared by the above method, 150 μL of the live bacteria (1×10⁸ CFU) of the strain of each Akkermansia phylogroup (EB-AMDK19 and EB-AMDK39) was orally administered to each experimental group over a total of two days once a day. After 17 days of the administration, 150 μL of the live bacteria (1×10⁸ CFU) of the strain of the phylogroups (EB-AMDK19 and EB-AMDK39) in the exclusion relationship was orally administered to each experimental group over a total of two days once a day. After the oral administration, fresh feces for each experimental group were periodically collected and stored in a −80° C. freezer in order to confirm changes in the gut level for each Akkermansia phylogroup. Changes in the gut level for each Akkermansia phylogroup according to the cross-administration of the Akkermansia phylogroups were confirmed by performing quantitative PCR on gDNA extracted from fresh feces collected for each experimental group at each time point after the cross-administration of the Akkermansia phylogroups, and the results are shown in FIGS. 10a and 10b.

TABLE 10

| Experimental | |
|---|---|
| groups | Administration information |
| Experimental | Group of administration of EB-AMDK 19 live bacteria |
| Group I | (1 × 10⁸ CFU) representing AmIb phylogroup, and after |
| | 17 days of AmIb administration, cross-administration |
| | of EB-AMDK39 live bacteria (1 × 10⁸ CFU) |
| | representing AmII phylogroup |
| Experimental | Group of administration of EB-AMDK39 live bacteria |
| Group II | (1 × 10⁸ CFU) representing AmII phylogroup, and after |
| | 17 days of AmII administration, cross-administration |
| | of EB-AMDK19 live bacteria (1 × 10⁸ CFU) |
| | representing AmIb phylogroup |

Referring to FIG. 10a, it may be confirmed through values of genome equivalents (log 10)/g feces specific to the AmII phylogroup primer that EB-AMDK39 representing the AmII phylogroup is administered first and then settled in the gut. As the gut Akkermansia phylogroup was specified as the AmII phylogroup (17 days after the administration of the AmII phylogroup), EB-AMDK19 belonging to the AmIb phylogroup was administered and the changes in the gut Akkermansia phylogroup according to the cross-administration was observed. As a result, the values of genome equivalents (log 10)/g feces specific to the AmII phylogroup primer was retained until the end of the experiment and the values of genome equivalents (log 10)/g feces specific to the AmIb phylogroup primer was not increased, and thus it may be confirmed that the gut Akkermansia dominant species is retained as the AmII phylogroup.

Referring to FIG. 10b, it may be confirmed through values of genome equivalents (log 10)/g feces specific to the AmIb phylogroup primer that EB-AMDK19 representing the AmIb phylogroup is administered first and then settled in the gut. As the gut Akkermansia phylogroup was specified as the AmI phylogroup (17 days after the administration of the AmIb phylogroup), EB-AMDK39 belonging to the AmII phylogroup was administered and the changes in the gut Akkermansia phylogroup according to the cross-administration was observed. As a result, the values of genome equivalents (log 10)/g feces specific to the AmIb phylogroup primer was retained until the end of the experiment and the values of genome equivalents (log 10)/g feces specific to the AmII phylogroup primer was not increased, and thus it may be confirmed that the gut Akkermansia dominant species is retained as the AmIb phylogroup. When these results were combined, it was confirmed that it was not easy for Akkermansia bacteria in other exogenous phylogroups to settle while a specific Akkermansia phylogroup settled in the gut.

The above-described examples are to be understood in all aspects as illustrative and not restrictive. The scope of the present invention is defined by the following claims rather than the detailed description. It shall be understood that all modifications or changes in forms conceived from the claims are included in the scope of the present invention.

1. A method for predicting a therapeutic response of a patient to biotherapeutics, the method comprising:
(a) confirming the distribution of target bacteria for each strain or phylogroup through gut microbiota analysis of the patient;
(b) identifying whether there is a competitive exclusion relationship between a strain or phylogroup identified as a gut-dominant species of the patient in the previous step and a target strain or phylogroup; and
(c) determining that when the strain or phylogroup identified as the gut-dominant species and the strain or phylogroup of the target bacteria are identified to have a competitive exclusion relationship in the previous step, the therapeutic response of the patient to the biotherapeutics including the corresponding strain or phylogroup is low.

2. The method of claim 1, wherein the gut microbiota analysis comprises performing quantitative PCR (qPCR) on DNA extracted from a fecal sample of the patient using a primer pair or probe specific to the sodium ion-translocating decarboxylase subunit beta gene of bacteria of a specific strain or phylogroup.

3. The method of claim 2, wherein the primer is an AmIa-specific primer having at least 75% sequence homology to the sequence of SEQ ID NO: 1 or SEQ ID NO: 2;
an AmIb-specific primer having at least 75% sequence homology to the sequence of SEQ ID NO: 1 or SEQ ID NO: 2;
an AmII-specific primer having at least 75% sequence homology to the sequence of SEQ ID NO: 5 or SEQ ID NO: 6; or
an AmIV-specific primer having at least 75% sequence homology to the sequence of SEQ ID NO: 7 or SEQ ID NO: 8.

4. The method of claim 1, wherein the gut microbiota analysis comprises identifying a phylogroup and confirming the distribution using a phylogroup-specific genetic identification region specific to the 16S rRNA gene of bacteria of a specific strain or phylogroup on DNA extracted from a fecal sample of the patient.

5. The method of claim 4, wherein the Akkermansia phylogroup-specific identification region is:
phylogroup identification region 1 having at least 75% sequence homology to the gene sequence of the AmI-specific identification region of SEQ ID NO: 9, the gene sequence of the AmII-specific identification region of SEQ ID NO: 10, or the gene sequence of the AmIV-specific identification region of SEQ ID NO: 11;
phylogroup identification region 2 having at least 75% sequence homology to the gene sequence of the AmI-specific identification region of SEQ ID NO: 12, the gene sequence of the AmII-specific identification region of SEQ ID NO: 13, or the gene sequence of the AmIV-specific identification region of SEQ ID NO: 14; or
phylogroup identification region 3 having at least 75% sequence homology to the gene sequence of the AmI-specific identification region of SEQ ID NO: 15, the gene sequence of the AmII-specific identification region of SEQ ID NO: 16, or the gene sequence of the AmIV-specific identification region of SEQ ID NO: 17.

6. The method of claim 1, wherein the identifying of the presence or absence of a competitive exclusion relationship comprises treating the target strain or phylogroup with the culture supernatant of the strain or phylogroup identified as the gut-dominant species of the patient to determine whether the growth of the target strain or phylogroup is inhibited.

7. The method of claim 1, wherein the therapeutic bacteria are Akkermansia.

8. The method of claim 7, wherein the Akkermansia has phylogroups AmIa, AmIb, AmII, and AmIV, and has competitive exclusion relationship between these phylogroups that the growth of the phylogroups AmIa and AmIb is inhibited by phylogroups AmII and AmIV, but does not inhibit the growth of phylogroups AmII and AmIV, and the growth of the phylogroup AmII is inhibited by phylogroup AmIV, but does not inhibit the growth of phylogroup AmIV.

9. The method of claim 1, wherein the patient is a patient with a metabolic disorder, an inflammatory disease, an atopic disease, a cranial nerve disease, or cancer.

10. The method of claim 1, wherein the patient is a patient with a metabolic disorder selected from the group consisting of metabolic syndrome, insulin-deficiency, insulin-resistance related disorders, diabetes mellitus, glucose intolerance, abnormal lipid metabolism, atherosclerosis, hypertension, pre-eclampsia, stroke, non-alcoholic fatty liver disease, hyperglycemia, hepatic steatosis, dyslipidemia, inflammatory diseases including Crohn's disease, ulcerative colitis, and irritable bowel syndrome, cardiovascular diseases, cerebrovascular diseases, peripheral vascular diseases, high cholesterol, elevated triglyceride, asthma, atopic dermatitis, sleep apnea, osteoarthritis, neurodegeneration, gallbladder diseases, and atherogenic dyslipidemia.

11. A marker composition for predicting a therapeutic response of a patient with a metabolic disorder to biotherapeutics comprising Akkermansia sp. bacteria, wherein the marker composition comprises:
an AmIa-specific primer having at least 75% sequence homology to the sequence of SEQ ID NO: 1 or SEQ ID NO: 2;
an AmIb-specific primer having at least 75% sequence homology to the sequence of SEQ ID NO: 3 or SEQ ID NO: 4;
an AmII-specific primer having at least 75% sequence homology to the sequence of SEQ ID NO: 5 or SEQ ID NO: 6; or
an AmIV-specific primer having at least 75% sequence homology to the sequence of SEQ ID NO: 7 or SEQ ID NO: 8.

12. A marker composition for predicting a therapeutic response of a patient with a metabolic disorder to biotherapeutics comprising Akkermansia sp. bacteria, wherein the marker composition comprises: phylogroup identification region 1 having at least 75% sequence homology to the gene sequence of the AmI-specific identification region of SEQ ID NO: 9, the gene sequence of the AmII-specific identification region of SEQ ID NO: 10, or the gene sequence of the AmIV-specific identification region of SEQ ID NO: 11;
phylogroup identification region 2 having at least 75% sequence homology to the gene sequence of the AmI-specific identification region of SEQ ID NO: 12, the gene sequence of the AmII-specific identification region of SEQ ID NO: 13, or the gene sequence of the AmIV-specific identification region of SEQ ID NO: 14; or
phylogroup identification region 3 having at least 75% sequence homology to the gene sequence of the AmI-specific identification region of SEQ ID NO: 15, the gene sequence of the AmII-specific identification region of SEQ ID NO: 16, or the gene sequence of the AmIV-specific identification region of SEQ ID NO: 17.

13. A method for predicting a therapeutic response of a patient to biotherapeutics, the method comprising:
(a) confirming the distribution of Akkermansia sp. bacteria to be used as biotherapeutics for each strain or phylogroup through gut microbiota analysis of the patient;
(b) identifying whether there is a competitive exclusion relationship between the Akkermansia sp. strain or phylogroup identified as a gut-dominant species in the previous step and the target Akkermansia sp. strain or phylogroup and
(c) determining that when the Akkermansia sp. phylogroup identified as the gut-dominant species and the target Akkermansia sp. bacteria are identified to have a competitive exclusion relationship in the previous step, the therapeutic response of the patient to the biotherapeutics including the corresponding Akkermansia sp. strain or phylogroup is low.

14. The method of claim 13, wherein said Akkermansia has phylogroups AmIa, AmIb, AmII, and AmIV, and has competitive exclusion relationship between these phylogroups that the growth of the phylogroups AmIa and AmIb is inhibited by phylogroups AmII and AmIV, but does not inhibit the growth of phylogroups AmII and AmIV, and the growth of the phylogroup AmII is inhibited by phylogroup AmIV, but does not inhibit the growth of phylogroup AmIV.

15. A method for treating patients with various diseases including a metabolic disorder, the method comprising:
(a) confirming the distribution of target bacteria for each strain or phylogroup through gut microbiota analysis of the patient;
(b) identifying whether there is a competitive exclusion relationship between a strain or phylogroup identified as a gut-dominant species of the patient in the previous step and a target strain or phylogroup; and
(c) when the strain or phylogroup identified as the dominant species and the strain or phylogroup of the target bacteria are identified to have no competitive exclusion relationship in the previous step, selecting, as a treatment option, biotherapeutics including the corresponding strain or phylogroup to administer the biotherapeutics to the patient.

16. The method of claim 15, wherein the metabolic disorder is selected from the group consisting of metabolic syndrome, insulin-deficiency or insulin-resistance related disorders, diabetes mellitus, glucose intolerance, abnormal lipid metabolism, atherosclerosis, hypertension, pre-eclampsia, stroke, non-alcoholic fatty liver disease, hyperglycemia, hepatic steatosis, dyslipidemia, inflammatory diseases including Crohn's disease, ulcerative colitis, and irritable bowel syndrome, cardiovascular diseases, cerebrovascular diseases, peripheral vascular diseases, high cholesterol, elevated triglyceride, asthma, atopic dermatitis, sleep apnea, osteoarthritis, neurodegeneration, gallbladder diseases, and atherogenic dyslipidemia.

17. The method of claim 15, wherein the biotherapeutics comprises Akkermansia, and the Akkermansia has phylogroups AmIa, AmIb, AmII, and AmIV, and the phylogroups AmI and AmII, and the phylogroups AmIa and AmIb have competitive exclusion relationships, respectively.


SEQUENCE LISTING (23 sequences)

SEQ ID NO: 1 - DNA, 15 bp, synthetic construct
  source 1..15: mol_type = other DNA
  misc_feature 1..15: note = AmIa primer (forward)
cgcttcagcaggctc

SEQ ID NO: 2 - DNA, 17 bp, synthetic construct
  source 1..17: mol_type = other DNA
  misc_feature 1..17: note = AmIa primer (reverse)
ggttcattgccgttgtc

SEQ ID NO: 3 - DNA, 16 bp, synthetic construct
  source 1..16: mol_type = other DNA
  misc_feature 1..16: note = AmIb primer (forward)
atgtggctcctctgca

SEQ ID NO: 4 - DNA, 15 bp, synthetic construct
  source 1..15: mol_type = other DNA
  misc_feature 1..15: note = AmIb primer (reverse)
aggtccacgggaaca

SEQ ID NO: 5 - DNA, 16 bp, synthetic construct
  source 1..16: mol_type = other DNA
  misc_feature 1..16: note = AmII primer (forward)
ttggcgcattaattgc

SEQ ID NO: 6 - DNA, 21 bp, synthetic construct
  source 1..21: mol_type = other DNA
  misc_feature 1..21: note = AmII primer (reverse)
tccgtgtcaaagtagttgact

SEQ ID NO: 7 - DNA, 19 bp, synthetic construct
  source 1..19: mol_type = other DNA
  misc_feature 1..19: note = AmIV primer (forward)
gacttctccaatgtcagcg

SEQ ID NO: 8 - DNA, 16 bp, synthetic construct
  source 1..16: mol_type = other DNA
  misc_feature 1..16: note = AmIV primer (reverse)
tcccttgctgacctgc

SEQ ID NO: 9 - DNA, 91 bp, Akkermansia muciniphila
  source 1..91: mol_type = other DNA
  misc_feature 1..91: note = (91 bp) AmI primer
cccgagagcgggatagccctgggaaactgggattaataccgcatagtatcgaaagattaaagcagcaatgcgcttggggatgggctcgcgg

SEQ ID NO: 10 - DNA, 91 bp, Akkermansia muciniphila
  source 1..91: mol_type = other DNA
  misc_feature 1..91: note = (91 bp) AmII primer
cccaagagtgggatagccccgggaaactgggattaataccgcataaaatcgcaagattaaagcagcaatgcgcttggggatgggctcgcgt

SEQ ID NO: 11 - DNA, 91 bp, Akkermansia muciniphila
  source 1..91: mol_type = other DNA
  misc_feature 1..91: note = (91 bp) AmIV primer
tcttagtgggggatagccctgggaaaccgggattaataccgcatacgattgaaagatcaaagcagcaatgcgctaggagatgggctcgcgg

SEQ ID NO: 12 - DNA, 46 bp, Akkermansia muciniphila
  source 1..46: mol_type = other DNA
  misc_feature 1..46: note = (46 bp) AmI primer
tgtttcgtaagtcgtgtgtgaaaggcgcgggctcaacccgcggacg

SEQ ID NO: 13 - DNA, 46 bp, Akkermansia muciniphila
  source 1..46: mol_type = other DNA
  misc_feature 1..46: note = (46 bp) AmII primer
ggtttcgtaagtcgtgtgtgaaaggcgggggctcaacccccggact

SEQ ID NO: 14 - DNA, 46 bp, Akkermansia muciniphila
  source 1..46: mol_type = other DNA
  misc_feature 1..46: note = (46 bp) AmIV primer
tgtttcgtaagtcgtgtgtgaaaggcaggggctcaacccctggatt

SEQ ID NO: 15 - DNA, 12 bp, Akkermansia muciniphila
  source 1..12: mol_type = other DNA
  misc_feature 1..12: note = (12 bp) AmI primer
gcacgtgaaggt

SEQ ID NO: 16 - DNA, 12 bp, Akkermansia muciniphila
  source 1..12: mol_type = other DNA
  misc_feature 1..12: note = (12 bp) AmII primer
gcacgtaaaggt

SEQ ID NO: 17 - DNA, 12 bp, Akkermansia muciniphila
  source 1..12: mol_type = other DNA
  misc_feature 1..12: note = (12 bp) AmIV primer
tcgagtaatgtc

SEQ ID NO: 18 - DNA, 253 bp, synthetic construct
  source 1..253: mol_type = other DNA
  misc_feature 1..253: note = AmIa primer
cgcttcagcaggctccctccaaggtgccggccaatctgaccacgcctgaatcccgtgcccagtatcaggaaatcatgcagcagcccatgcaggtttaccccggcagccagctgaccgtttccaagatcaagtccgtgcgggaatcccaggaaaaagcaaaagctgacgcggcccgcctgggcgacgacagcctgacggtggaccccaacctgaaggatttccagaacgtgacggaagacaacggcaatgaacc

SEQ ID NO: 19 - DNA, 358 bp, synthetic construct
  source 1..358: mol_type = other DNA
  misc_feature 1..358: note = AmIb primer
atgtggctcctctgcagcagacgcccgccaaggtgccggccaatctgaccacgccggaagcccgcgcccagtatctggaaaccatgcagcagcccatgcaggtttatcccggcagccagctgaccgtttccaaaatcaagtccgtgcgggaatcccaggaaaaagccaaagctgacgccgcccgcctgggagacgacagtctgacggtggatcccaacctgaaggacttccagaacgtgacggaggacaacggcaatgagccggtcttcctgctcacgaacggagaaggaaccaccgtcgtccgccagcagggcgtcaattactttgacaccagcggcaaccgtgttcccgtggacct

SEQ ID NO: 20 - DNA, 466 bp, synthetic construct
  source 1..466: mol_type = other DNA
  misc_feature 1..466: note = AmII primer
ttggcgcattaattgccaacattccggacaacggaatgctcatcacccagctgaaccagcaggtcatctcctccaataaggcgggggaagtgaccgccacgtccctcaacaacgtgggctacctgcgcgttcacgtggccccgctccagcaggcccccgtcaaggtgccggccaacctgaccacgcctgaagcgcgcgcccagtatctggcaaacatggaacagcccatgcaggtttaccccggcagcccgctgaccgtctcccagatcaagcccgtgcgggaatcccaggaaaaagccaaggctgacgccgcccttctgggggacgacagcctgacggtggaccccaacctgaaggacttccacaacgtgacggaagcggacgccaacgatccggtgtacctgctcacgaacggtggagtcaccaccgttctgcgccagaagggagtcaactactttgacacgga

SEQ ID NO: 21 - DNA, 1078 bp, Akkermansia muciniphila
  source 1..1078: mol_type = genomic DNA
  misc_feature 1..1078: note = AmI
ggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggccc

SEQ ID NO: 22 - DNA, 1078 bp, Akkermansia muciniphila
  source 1..1078: mol_type = genomic DNA
  misc_feature 1..1078: note = AmII
ggcgcacgggtgagtaacacgtgagtaacctgcccccaagagtgggatagccccgggaaactgggattaataccgcataaaatcgcaagattaaagcagcaatgcgcttggggatgggctcgcgtcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtaaaggtggggactctggcgagactgcccagatcaactgggaggaaggtggggacgacgtcaggtcagtatggccc

SEQ ID NO: 23 - DNA, 1078 bp, Akkermansia muciniphila
  source 1..1078: mol_type = genomic DNA
  misc_feature 1..1078: note = AmIV
ggcgcacgggtgagtaacacgtgagtaacctgcctcttagtgggggatagccctgggaaaccgggattaataccgcatacgattgaaagatcaaagcagcaatgcgctaggagatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggcaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcaggggctcaacccctggattgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccatcgagtaatgtcggggactctggcgagactgcccagatcaactgggaggaaggtggggacgacgtcaggtcagtatggccc